(19) United States
(12) Patent Application Publication
(10) Pub. No.: US 2004/0038344 A1
(43) Pub. Date: Feb. 26, 2004

(54) Integrin protein

(76) Inventor: Anne E. Cress, Tucson, AZ (US)

Correspondence Address: FISH & RICHARDSON PC, 225 FRANKLIN ST, BOSTON, MA, 02110, US

(21) Appl. No.: 10/383,183

(22) Filed: Mar. 6, 2003

Related U.S. Application Data
(60) Provisional application No. 60/362,430, filed on Mar. 6, 2002.

(51) Int. Cl. C12P 21/02; C12N 5/06; C07K 14/74; C07H 21/04

(52) U.S. Cl. 435/69.1; 435/320.1; 435/325; 530/350; 536/23.5

(57) ABSTRACT

The compositions and methods of the invention provide isolated N-terminally truncated alpha6 integrin polypeptides, also called “alpha6 integrin variants,” and methods of making the polypeptides.

### RELATED APPLICATIONS

This application claims priority to U.S. provisional application 60/362,430, filed on Mar. 6, 2002, the contents of which are incorporated herein by reference.

### GOVERNMENT SUPPORT

The work described herein was carried out, at least in part, using funds from a federal grant, number NCI/NIH CA 56666. The government may, therefore, have certain rights in the invention.

### TECHNICAL FIELD

This invention relates to cell surface and extracellular proteins.

### BACKGROUND

Integrins are cell surface receptors that are involved in cell-matrix adhesion and signaling. The alpha6 integrin is a laminin receptor and contains 1050 amino acids, present in the form of a heavy (110-kDa) and a light (30-kDa) chain linked by a disulfide bond. The heavy chain of alpha6 integrin contains an 875-amino acid extracellular region and interacts with a beta subunit to form a heterodimer. Integrin subunits typically contain seven weak sequence repeats in the N-terminal region that are thought to be important for ligand binding and have been predicted to fold cooperatively into a single beta-propeller domain with seven beta sheets.

Two alternatively spliced forms of alpha6 integrin, called alpha6A and alpha6B, are identical in their heavy chains and differ in their light chain compositions. The light chains of the alpha6 integrin isoforms are identical in their 170 amino acid extracellular domains and also in their transmembrane domains. The two isoforms differ in their cytoplasmic regions. Because of their identical extracellular domains, alpha6A and alpha6B integrins can each pair with either the betal or the beta4 subunit, and each isoform is found on a variety of normal cell types including platelets, epithelia, endothelia, proximal and distal tubules of the kidney, Schwann and perineural cells, and lymphoid follicles.

### SUMMARY OF THE INVENTION

Alpha6p integrin is a naturally occurring truncated form of the alpha6 integrin. Alpha6p migrates as a 70 kDa protein on an SDS-PAGE gel (SDS-polyacrylamide gel electrophoresis). Integrin alpha6p is believed to contain amino acid sequences corresponding to the C-terminal region of alpha6, which corresponds to the region containing the extracellular “stalk,” the transmembrane sequence, and the cytoplasmic tail of the full-length alpha6 integrin. The compositions and methods of the invention provide isolated N-terminally truncated alpha6 integrin polypeptides, also referred to herein as “alpha6 integrin variants,” and methods of making and using the isolated polypeptides.

The invention includes, inter alia, an isolated polypeptide that includes a truncated alpha6 integrin. A “truncated alpha6 integrin,” also referenced herein as an “alpha6 integrin variant” or “truncated alpha6 polypeptide,” is an alpha6 integrin that does not include an amino acid sequence from the N-terminus of the alpha6 integrin. For example, a sequence corresponding to at least amino acids 1-516 of SEQ ID NO:1 is not included in the truncated alpha6 integrin. In other words, the amino acids encoded by exons 1-11 of the alpha6 cDNA (SEQ ID NO:2; GenBank Accession No. AH008066.1; see Table 1) are not present in the truncated alpha6 integrin. The sequence of SEQ ID NO:2 is as follows.

ATGGCCGCCG CCGGGCAGCT GTGCTTGCTC TACCTGTCGG
CGGGGCTCCT GTCCCGGCTC GGCGCAGCCT TCAACTTGGA
CACTCGGGAG GACAACGTGA TCCGGAAATA TGGAGACCCC
GGGAGCCTCT TCGGCTTCTC GCTGGCCATG CACTGGCAAC
TGCAGCCCGA GGACAAGCGG CTGTTGCTCG TGGGGGCCCC
GCGGGCAGAA GCGCTTCCAC TGCAGAGAGC CAACAGAACG
GGAGGGCTGT ACAGCTGCGA CATCACCGCC CGGGGGCCAT
GCACGCGGAT CGAGTTTGAT AACGATGCTG ACCCCACGTC
AGAAAGCAAG GAAGATCAGT GGATGGGGGT CACCGTCCAG
AGCCAAGGTC CAGGGGGCAA GGTCGTGACA TGTGCTCACC
GATATGAAAA AAGGCAGCAT GTTAATACGA AGCAGGAATC
CCGAGACATC TTTGGGCGGT GTTATGTCCT GAGTCAGAAT
CTCAGGATTG AAGACGATAT GGATGGGGGA GATTGGAGCT
TTTGTGATGG GCGATTGAGA GGCCATGAGA AATTTGGCTC
TTGCCAGCAA GGTGTAGCAG CTACTTTTAC TAAAGACTTT
CATTACATTG TATTTGGAGC CCCGGGTACT TATAACTGGA
AAGGGATTGT TCGTGTAGAG CAAAAGAATA ACACTTTTTT
TGACATGAAC ATCTTTGAAG ATGGGCCTTA TGAAGTTGGT
GGAGAGACTG AGCATGATGA AAGTCTCGTT CCTGTTCCTG
CTAACAGTTA CTTAGGTTTT TCTTTGGACT CAGGGAAAGG
TATTGTTTCT AAAGATGAGA TCACTTTTGT ATCTGGTGCT
CCCAGAGCCA ATCACAGTGG AGCCGTGGTT TTGCTGAAGA
GAGACATGAA GTCTGCACAT CTCCTCCCTG AGCACATATT
CGATGGAGAA GGTCTGGCCT CTTCATTTGG CTATGATGTG
GCGGTGGTGG ACCTCAACAA GGATGGGTGG CAAGATATAG
TTATTGGAGC CCCACAGTAT TTTGATAGAG ATGGAGAAGT
TGGAGGTGCA GTGTATGTCT ACATGAACCA GCAAGGCAGA
TGGAATAATG TGAAGCCAAT TCGTCTTAAT GGAACCAAAG
ATTCTATGTT TGGCATTGCA GTAAAAAATA TTGGAGATAT
TAATCAAGAT GGCTACCCAG ATATTGCAGT TGGAGCTCCG
TATGATGACT TGGGAAAGGT TTTTATCTAT CATGGATCTG
CAAATGGAAT AAATACCAAA CCAACACAGG TTCTCAAGGG
TATATCACCT TATTTTGGAT ATTCAATTGC TGGAAACATG
GACCTTGATC GAAATTCCTA CCCTGATGTT GCTGTTGGTT
CCCTCTCAGA TTCAGTAACT ATTTTCAGAT CCCGGCCTGT
GATTAATATT CAGAAAACCA TCACAGTAAC TCCTAACAGA
ATTGACCTCC GCCAGAAAAC AGCGTGTGGG GCGCCTAGTG
GGATATGCCT CCAGGTTAAA TCCTGTTTTG AATATACTGC
TAACCCCGCT GGTTATAATC CTTCAATATC AATTGTGGGC
ACACTTGAAG CTGAAAAAGA AAGAAGAAAA TCTGGGCTAT
CCTCAAGAGT TCAGTTTCGA AACCAAGGTT CTGAGCCCAA
ATATACTCAA GAACTAACTC TGAAGAGGCA GAAACAGAAA
GTGTGCATGG AGGAAACCCT GTGGCTACAG GATAATATCA
GAGATAAACT GCGTCCCATT CCCATAACTG CCTCAGTGGA
GATCCAAGAG CCAAGCTCTC GTAGGCGAGT GAATTCACTT
CCAGAAGTTC TTCCAATTCT GAATTCAGAT GAACCCAAGA
CAGCTCATAT TGATGTTCAC TTCTTAAAAG AGGGATGTGG
AGACGACAAT GTATGTAACA GCAACCTTAA ACTAGAATAT
AAATTTTGCA CCCGAGAAGG AAATCAAGAC AAATTTTCTT
ATTTACCAAT TCAAAAAGGT GTACCAGAAC TAGTTCTAAA
AGATCAGAAG GATATTGCTT TAGAAATAAC AGTGACAAAC
AGCCCTTCCA ACCCAAGGAA TCCCACAAAA GATGGCGATG
ACGCCCATGA GGCTAAACTG ATTGCAACGT TTCCAGACAC
TTTAACCTAT TCTGCATATA GAGAACTGAG GGCTTTCCCT
GAGAAACAGT TGAGTTGTGT TGCCAACCAG AATGGCTCGC
AAGCTGACTG TGAGCTCGGA AATCCTTTTA AAAGAAATTC
AAATGTCACT TTTTATTTGG TTTTAAGTAC AACTGAAGTC
ACCTTTGACA CCCCATATCT GGATATTAAT CTGAAGTTAG
AAACAACAAG CAATCAAGAT AATTTGGCTC CAATTACAGC
TAAAGCAAAA GTGGTTATTG AACTGCTTTT ATCGGTCTCG
GGAGTTGCTA AACCTTCCCA GGTGTATTTT GGAGGTACAG
TTGTTGGCGA GCAAGCTATG AAATCTGAAG ATGAAGTGGG
AAGTTTAATA GAGTATGAAT TCAGGGTAAT AAACTTAGGT
AAACCTCTTA CAAACCTCGG CACAGCAACC TTGAACATTC
AGTGGCCAAA AGAAATTAGC AATGGGAAAT GGTTGCTTTA
TTTGGTGAAA GTAGAATCCA AAGGATTGGA AAAGGTAACT
TGTGAGCCAC AAAAGGAGAT AAACTCCCTG AACCTAACGG
AGTCTCACAA CTCAAGAAAG AAACGGGAAA TTACTGAAAA
ACAGATAGAT GATAACAGAA AATTTTCTTT ATTTGCTGAA
AGAAAATACC AGACTCTTAA CTGTAGCGTG AACGTGAACT
GTGTGAACAT CAGATGCCCG CTGCGGGGGC TGGACAGCAA
GGCGTCTCTT ATTTTGCGCT CGAGGTTATG GAACAGCACA
TTTCTAGAGG AATATTCCAA ACTGAACTAC TTGGACATTC
TCATGCGAGC CTTCATTGAT GTGACTGCTG CTGCCGAAAA
TATCAGGCTG CCAAATGCAG GCACTCAGGT TCGAGTGACT
GTGTTTCCCT CAAAGACTGT AGCTCAGTAT TCGGGAGTAC
CTTGGTGGAT CATCCTAGTG GCTATTCTCG CTGGGATCTT
GATGCTTGCT TTATTAGTGT TTATACTATG GAAGTGTGGT
TTCTTCAAGA GAAATAAGAA AGATCATTAT GATGCCACAT
ATCACAAGGC TGAGATCCAT GCTCAGCCAT CTGATAAAGA
GAGGCTTACT TCTGATGCAT AG

TABLE 1
Exon boundaries as defined by nucleotide numbers in SEQ ID NO: 2
and amino acid residues in SEQ ID NO: 1.

| | Nucleotides in | Amino Acids in |
|---|---|---|
| Exon | SEQ ID NO: 2 | SEQ ID NO: 1 |
| 1 | <1-182 | 1-60 |
| 2 | 183-307 | 61-102 |
| 3 | 308-387 | 103-128 |
| 4 | 388-643 | 129-214 |
| 5 | 644-775 | 215-258 |
| 6 | 776-986 | 259-329 |
| 7 | 987-1180 | 330-393 |
| 8 | 1181-1269 | 394-423 |
| 9 | 1270-1388 | 424-463 |
| 10 | 1389-1487 | 464-496 |
| 11 | 1488-1549 | 497-516 |
| 12 | 1550-1710 | 517-570 |
| 13 | 1711-1854 | 571-618 |
| 14 | 1855-1970 | 619-656 |
| 15 | 1971-2160 | 657-719 |
| 16 | 2161-2244 | 720-747 |
| 17 | 2245-2324 | 748-774 |
| 18 | 2325-2402 | 775-799 |
| 19 | 2403-2505 | 800-832 |
| 20 | 2506-2679 | 833-889 |
| 21 | 2680-2778 | 890-922 |
| 22 | 2779-2889 | 923-959 |
| 23 | 2890-2988 | 960-992 |
| 24 | 2989-3114 | 993-1034 |
| 25 | 2115->3222 | 1035-1073 |

SEQ ID NO:1 represents the amino acid sequence of an exemplary full-length alpha6 integrin protein. The N-terminal amino acid of the truncated alpha6 integrin can begin with an amino acid that corresponds to an amino acid between residues 516-597, 570-597, or 587-597 of SEQ ID NO:1, e.g., amino acid 587, 588, 589, 590, 591, 592, 593, 594, 595, or 596. In other words, the N-terminal amino acid of the truncated alpha6 integrin can begin with an amino acid encoded by a sequence of exons 12 or 13 of SEQ ID NO:2. The N-terminus of the truncated aplha6 integrin polypeptide can begin with the amino acid sequence arginine-valine-asparagine (RVN), RRVN (SEQ ID NO:4), RRRVN (SEQ ID NO:5), SRRRVN (SEQ ID NO:6), or SSRRRVN (SEQ ID NO:7). The N terminus of the truncated alpha6 integrin can include at least the amino acid sequence RVN of SEQ ID NO:1, but the truncated alpha6 integrin excludes amino acids 1-516 of alpha 6 integrin. Amino acids 1-516 are encoded by exons 1-11 (nucleotides 1-1856) of SEQ ID NO:2. In certain embodiments, the truncated alpha6 integrin includes only the extracellular region (the ectodomain), e.g., a sequence that terminates at an amino acid corresponding to an amino acid within ten amino acids of 1050 of SEQ ID NO:1.

In one aspect, the isolated polypeptide of the invention is a C-terminal alpha6 integrin fragment that includes at least an amino acid sequence corresponding to amino acids 596-1050 or 596-1073 of the full-length alpha6 integrin (e.g., SEQ ID NO:1) and does not include a region corresponding to at least amino acids 1-516 of the full-length alpha6 integrin (e.g., SEQ ID NO:1). The C-terminal alpha6 integrin fragment can be located within a larger polypeptide sequence. For example, the fragment can be attached to a heterologous, non-integrin sequence, e.g., an N-terminal or C-terminal heterologous sequence, e.g., a tag or a domain of a protein and so forth. For example, the ectodomain of the truncated alpha6 integrin can be fused to a C-terminal tag, e.g., a hexahistidine sequence, an Fc domain, a GST protein, maltose binding protein, and so forth or to a reporter sequence, e.g., an enzyme such as alkaline phosphatase. In another example the ectodomain of the truncated alpha6 integrin is fused to a heterologous transmembrane sequence, a myristylation signal (e.g., the DAF signal), a viral protein, or a transmembrane sequence and a heterologous cytoplasmic domain.

The truncated alpha6 integrin can have the same N-terminus as a naturally-occurring truncated alpha6 integrin or the same sequence as a naturally-occurring truncated alpha6 integrin. Exemplary naturally-occurring truncated alpha6 integrins are found on cells of the prostate tumor cell lines DU145H (Rabinovitz et al., Clin. Exp. Metastasis 13:481-491, 1995), PC3 (Tran et al., Am. J. Pathol. 155:787-798), and LnCap; the colon cancer cell line SW480, and the normal immortalized keratinocyte cell line HaCat (Breitkrutz et al., Eur. J. Cell Biol. 75:273-286). The polypeptide can be a naturally occurring variant of the truncated alpha6 integrin, e.g., a variant that includes a sequence corresponding to a sequence within 517-1073, 571-1073, 588-1073, or 596-1073 of SEQ ID NO:1, e.g., substantially identical to amino acids 517-1073, 571-1073, 588-1073, or 596-1073 of SEQ ID NO:1. A sequence that is “substantially identical” is at least 85%, 90%, 95%, or 99% identical to an N-terminally truncated fragment of SEQ ID NO:1. For example, a sequence can differ by more than 5, 10, 20, 40, or 80 amino acids, but not more than 95 amino acids, from a sequence of the same length within SEQ ID NO:1, e.g., the sequence defined by amino acids 517-1073, 571-1073, 588-1073, or 596-1073 of SEQ ID NO:1. A sequence that “differs” from the specified sequence of SEQ ID NO:1, is a sequence in which an amino acid has been changed to a different amino acid, an amino acid has been deleted, or an amino acid has been added. In other embodiments, the truncated alpha6 polypeptide can be identical to an alpha6p amino acid sequence of a non-human animal, e.g., a non-human mammal (such as a, mouse, rat, pig, dog, or cat); a bird (such as a chicken); or an amphibian (such as a frog).

The truncated alpha6 integrin polypeptide can be a protease-resistant fragment of alpha6 integrin, and/or a naturally occurring fragment of alpha6 integrin. A “protease-resistant” polypeptide, as referenced herein, is as resistant or more resistant to degradation by proteases, such as kallikrein or plasmin, as alpha6 integrin, e.g., as determined by an assay described herein. A polypeptide of the invention can be resistant to intracellular and extracellular proteases in cell culture in vivo, when the polypeptide is presented on the surface of a cell. By “naturally occurring” is meant a polypeptide that is found in vivo, in nature, in an unmodified biological organism, such as a mammal or on a cultured cell from such an organism.

In one aspect, the truncated alpha6 polypeptide is alpha6p or a fragment of alpha6p. In another aspect, the truncated alpha6 polypeptide is isolated from a mammalian cell, such as a cell from a human, mouse, pig, dog, or cat; a cell from a bird, such as a chicken; or a cell from an amphibian, such as a frog. For example, the polypeptide can be isolated from a cell described herein.

In one embodiment, an isolated polypeptide of the invention can have an amino acid sequence that is at least 70, 80, 85, 90, 95, 96, 97, 98, 99, 99.5, or 100%, identical to a sequence of at least 100 or 400 amino acids within the amino acid sequence of amino acids 517-1073 of SEQ ID NO:1. Identity is measured by alignment of residues C-terminal of the truncation point.

An “isolated” or “purified” polypeptide or protein is substantially free of cellular material or other contaminating proteins from the cell or tissue source from which the protein is derived, or substantially free from chemical precursors or other chemicals when chemically synthesized. An isolated alpha6 polypeptide can be associated with another protein, e.g., a beta integrin subunit. “Substantially free” means that a preparation of truncated alpha6 integrin protein is at least 10% pure. In a preferred embodiment, the preparation of truncated alpha6 integrin protein has less than about 30%, 20%, 10% and more preferably 5% (by dry weight), of non-truncated alpha6 integrin protein (also referred to herein as a “contaminating protein”), or of chemical precursors. When the truncated alpha6 integrin protein or biologically active portion thereof is recombinantly produced, it is also preferably substantially free of culture medium, i.e., culture medium represents less than about 20%, more preferably less than about 10%, and most preferably less than about 5% of the volume of the protein preparation. The invention includes isolated or purified preparations of at least 0.01, 0.1, 1.0, and 10 milligrams in dry weight. It is also possible for the isolated or purified polypeptide to be in a membrane preparation, liposome, or an organic solvent. In still another example, it is possible to produce the polypeptide in a heterologous cell, e.g., in a cell that does not naturally produce the truncated alpha6 integrin.

A “non-essential” amino acid residue is a residue that can be altered from the wild-type sequence of truncated alpha6 integrin without abolishing or substantially altering a truncated alpha6 integrin activity. Preferably the alteration does not substantially alter the truncated alpha6 integrin activity, e.g., the activity is at least 20%, 40%, 60%, 70% or 80% of wild-type. An “essential” amino acid residue is a residue that, when altered from the wild-type sequence of truncated alpha6 integrin, results in abolishing a truncated alpha6 integrin activity such that less than 20% of the wild-type activity is present. For example, conserved amino acid residues in truncated alpha6 integrin may be particularly unamenable to alteration.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a truncated alpha6 integrin protein is preferably replaced with another amino acid residue from the same side chain family. Alternatively, in another embodiment, mutations can be introduced randomly along all or part of a truncated alpha6 integrin coding sequence, such as by saturation mutagenesis, and the resultant mutants can be screened for truncated alpha6 integrin biological activity to identify mutants that retain activity. Following mutagenesis a nucleic acid encoding the integrin, the encoded protein can be expressed recombinantly and the activity of the protein can be determined.

In one aspect, the truncated alpha6 polypeptide can bind an antibody that recognizes an epitope within amino acids 501-1073 of SEQ ID NO:1. For example, a truncated alpha6 polypeptide can bind to one or more of the antibodies GoH3, J1B5, 4F10, AA6A, and BQ16. The truncated alpha6 polypeptide will not sufficiently bind an antibody that recognizes an epitope located between amino acids 1 and 500 of SEQ ID NO:1. For example, the polyclonal antibody A33 (Sterk et al., J. Cell Biol. 149: 969-982, 2000) will not sufficiently bind a truncated alpha6 integrin of the invention. The phrase “does not substantially bind,” as used herein, means that the affinity of an antibody for the truncated alpha6 integrin is reduced by at least 70%, 80%, 90%, 95%, or more as compared to the binding affinity of an antibody that recognizes an alpha6 epitope that is within amino acids 596-1073 of SEQ ID NO:1. An “antibody,” as used herein, is an immunoglobulin molecule, or immunologically active portion thereof, i.e., an antigen-binding portion, that has a specific amino acid sequence by virtue of which it interacts only with the protein antigen that induced its synthesis or with a protein antigen closely related to it. Examples of immunologically active portions of immunoglobulin molecules include F(ab) and F(ab′)2 fragments that can be generated by treating the antibody with an enzyme such as pepsin. An antibody can be a polyclonal, monoclonal, recombinant, e.g., a chimeric or humanized, fully human, non-human, e.g., murine, or single chain antibody. An “epitope” is that part of the polypeptide against which a particular immune response is directed.

In one aspect, the invention features a heterologous nucleic acid that encodes a truncated alpha6 integrin variant (e.g., a variant described herein), but not a full length alpha6 integrin. The heterologous nucleic acid can include regulatory sequences, e.g., a promoter, untranslated leader sequence, a sequence encoding a signal sequence, a transcription termination sequence, introns, and so forth. The heterologous nucleic acid can further include a vector nucleic acid sequence, and in another aspect, the invention includes a host cell, e.g., a mammalian cell that contains a heterologous nucleic acid encoding a truncated alpha6 integrin variant. The heterologous nucleic acid that includes a sequence encoding a truncated alpha6 integrin, but not a full-length integrin can be used to provide cells that have the truncated alpha6 integrin on their cell surface, e.g., cells that do not produce the truncated alpha6 integrin unless the heterologous nucleic acid is expressed, e.g., induced.

In one embodiment, the invention provides a method for producing a truncated alpha6 integrin. The method includes culturing a host cell of the invention under conditions that allow the nucleic acid encoding the truncated alpha6 integrin to be expressed. The polypeptide can have an N-terminal amino acid within residues 516-597, 570-597, or 587-597 of SEQ ID NO:1, i.e., an N-terminal amino acid encoded by a sequence within exon 12 or 13 of SEQ ID NO:2. In one aspect, the truncated alpha6 integrin can be identical in sequence to a protease-resistant and naturally occurring integrin variant.

In one embodiment, the invention provides a method for evaluating a biological sample for a proliferative or epithelial disorder. A “biological sample” can be a tissue, cell, or fluid, from a subject, such as a mammal, e.g., a mouse or a human. According to the method, the sample can be provided, or obtained from a subject, such as a mammal, e.g., a human, and an N-terminally truncated alpha6 polypeptide can be detected. Detection of the polypeptide in the sample can indicate a proliferative disorder or an increased risk for a proliferative disorder. A “proliferative disorder” is a disorder characterized by abnormal cell division, e.g., uncontrolled cell division, e.g., a neoplasia. A cancer, e.g., an epithelial cancer (such as a melanoma), a prostate cancer, cervical cancer, breast cancer, colon cancer, or sarcoma, is an example of a proliferative disorder. Detection of the polypeptide in the sample can also indicate an epithelial disorder or an increased risk for an epithelial disorder. An “epithelial disorder” is a disorder that disrupts the structure and/or function of epithelial tissue, e.g., the skin. A blister and a disorder that causes blistering, such as an epidermolysis bullosa, are examples of epithelial disorders. Epithelial cancer is an example of an epithelial disorder that is also a proliferative disorder. To be at an “increased risk” means to be more likely to have the disorder than a biological sample in the general sample population.

In one embodiment, the method of detection of the truncated alpha6 integrin can include an immunoassay, such as an immunoprecipitation, ELISA, or in situ hybridization reaction; electrophoresis; mass spectrometry; and/or peptide sequencing.

In one embodiment, the sample is a tissue or a cell, or is obtained from a tissue, such as from epithelia, prostate, colon, breast, or lung. In another embodiment, the sample includes an endothelial cell.

In one embodiment, the invention provides a method for detecting a truncated alpha6 integrin in a biological sample, such as a tissue, cell, or fluid, from a subject, such as a mammal, e.g., a mouse or a human. The method includes surface-biotinylating cells of the sample, immunoprecipitating a truncated alpha6 protein from the sample. Immunoprecipitation can be performed using an antibody that recognizes an epitope that is encoded by a sequence within exons 12-25 of SEQ ID NO:2. Immunoprecipitated proteins can then be separated on an acrylamide gel by electrophoresis, and the proteins transferred from the gel to a membrane by a blotting method, e.g., by electrophoretic transfer. Transferred truncated alpha6p integrin can be detected by probing the membrane with streptavidin conjugated to a detectable label, such as peroxidase.

In another embodiment, the invention provides an alternative method for detecting a truncated alpha6 integrin in a biological sample. By this second method, the truncated alpha6 integrin is immunoprecipitated using an antibody that binds the truncated alpha6 integrin. This antibody can bind an epitope within the truncated integrin that is encoded by a sequence within exons 12-25 of SEQ ID NO:2. Following immunoprecipitation, the isolated protein(s) can be separated on an acrylamide gel, blotted to a membrane, and detected by Western blot analysis. For Western blot analysis, the primary antibody probe can be an antibody that recognizes an epitope encoded by a sequence within exons 12-25 of SEQ ID NO:2. Preferably, the primary antibody probe differs from the antibody used for the immunoprecipitation.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 80%, 85%, 90%, 95%, or 100% of the length of the reference sequence. For example, a polypeptide sequence of the invention would align to the reference amino acid sequence of alpha6 integrin that applies to the 14 C-terminal exons (exons 12-25). This sequence is represented by amino acids 517-1073 of SEQ ID NO:1, a total of 557 amino acids. A polypeptide of the invention can have an N-terminal amino acid within exons 12 or 13, and the amino acid sequence of the variant polypeptide would have at least 30%, preferably at least 40%, more preferably at least 50%, even more preferably at least 60%, and even more preferably at least 70%, 80%, or 90% of its amino acids aligned with the C-terminal region of SEQ ID NO:1. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The determination of percent identity between two amino acid sequences is accomplished using the BLAST® 2.0 program, which is available to the public from the National Center of Biotechnology Information (NCBI)(Bethesda Md.). Sequence comparison is performed using an ungapped alignment and using the default parameters (Blossom 62 matrix, gap existence cost of 11, per residue gapped cost of 1, and a lambda ratio of 0.85). The mathematical algorithm used in BLAST® programs is described in Altschul et al. (Nucleic Acids Res. 25:3389-3402, 1997). An amino acid in one sequence is said to “correspond” to an amino acid in another sequence if the amino acids are located in the same position in a BLAST alignment of the two sequences as described above.

It is also possible for a sequence encoding a truncated alpha6p integrin to hybridize to a corresponding integrin sequence, e.g., a sequence that includes SEQ ID NO:2, under particular conditions, e.g., low, medium, high, or very high stringency conditions. As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6×sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, useful methods and materials are described below. The materials, methods, and examples are illustrative only and not intended to be limiting. Other features and advantages of the invention will be apparent from the following detailed description, and from the claims.

### DESCRIPTION OF DRAWINGS

FIG. 1 is an image of a Western blot of alpha6 and alpha6p integrins immunoprecipitated from human cells. The DU145H cells were surface-biotinylated, and the alpha6 integrin was immunoprecipitated using either the GoH3, J1B5, AA6A, 4F10, or BQ16 antibodies, specific for human alpha6 integrin. The alpha5 integrin was precipitated from the lysate using the P1D6 antibody. The immunoprecipitations were analyzed using a 7.5% polyacrylamide gel under non-reducing conditions and the migration position of the biotinylated integrins are as indicated.

FIG. 2 is an image of a Western blot of a two-dimensional polyacrylamide gel. Surface-biotinylated proteins from DU145H cells were immunoprecipitated using the GoH3 antibody and were analyzed first by 7.5% polyacrylamide gel electrophoresis under non-reducing conditions. The resulting lane was excised from the gel and placed on the top of a second 7.5% polyacrylamide gel. The positions of the migration of the integrins in the first gel are indicated at the top of the figure. Electrophoresis was then performed under reducing conditions. The resulting migration of the heavy chain (HC) and light chain (LC) and the molecular masses are indicated. The asterisk indicates a biotinylated protein band that was variably seen and is of unknown identity.

FIG. 3 is an image of a pair of Western blots. FIG. 3A shows the results of coimmunoprecipitation experiments with A9, 439.9b, ASC3, or 3E1 antibodies, specific for human beta4 integrin, from surface-biotinylated HaCaT cells. FIG. 3B shows the results of coimmunoprecipitation experiments with a P4C10 antibody, specific for betal integrin, from surface-biotinylated DU145 cells and HaCaT cells. This Western blot also shows the results of a coimmunoprecipitation experiment with a J1B5 antibody, specific for alpha6 integrin, from the surface-biotinylated HaCaT cells. The immunoprecipitated proteins were analyzed using a 7.5% polyacrylamide gel under non-reducing conditions, and the migration positions of the biotinylated integrins are as indicated.

FIG. 4 is an image of a panel of Western blots showing the results of immunoprecipitation experiments with GoH3, J1B5, and P4C10 antibodies. The alpha6- and the beta1-containing integrins were immunoprecipitated from the lysates of human DU145H, HaCaT, and H69 cells with either anti-alpha6 integrin antibodies GoH3 or J1B5, or an anti-beta1 integrin antibody P4C10. The precipitated proteins were analyzed on a 7.5% polyacrylamide gel under non-reducing conditions followed by Western blot (WB) analysis with the alpha6A-specific antibodies 4E9G8 or AA6A, which are specific for the cytoplasmic domain, or the anti-alpha6 integrin antibody A33, which is specific for the N terminus of alpha6. The migration position of a biotinylated integrin standard from DU145H cells are as indicated. The samples shown in the middle panel were electrophoresed on a separate gel, and the molecular mass of the alpha6A band is indicated relative to the adjacent panels by a solid bar. The asterisk indicates a biotinylated protein band that was variably seen and is of unknown identity.

FIG. 5 is an image of a panel of Western blots showing the results of immunoprecipitation experiment with the GoH3 antibody. Alpha6-containing integrins were immunoprecipitated from the lysates of a normal, immortalized keratinocyte cell line (HaCaT) and a normal prostate epithelial cell line (PrEC), prostate cancer cell lines (PC3, PC3-N, and LnCaP), a breast cancer cell line (MCF-7), a colon carcinoma cell line (SW480), and a lung carcinoma cell line (H69). The immunoprecipitated proteins were analyzed on a 7.5% polyacrylamide gel under non-reducing conditions. The presence of alpha6p was detected by Western blot analysis using the AA6A antibody, specific for the human alpha6A light chain.

FIG. 6 shows exemplary amino acid sequences of exons 1-25 of the alpha6 integrin (SEQ ID NO:1). MALDI mass spectrometry and HPLC coupled to mass spectrometry identified 10 noncontinuous amino acid fragments from the alpha6p variant (boxed sequences). These corresponded exactly to sequences contained within exons 13-25. Five of nine putative glycosylation sites are retained within exons 13-25 and are indicated in bold and underlined type. Ten of 20 cysteine residues (indicated by closed circles) are retained within exons 13-25.

FIG. 7 is a schematic comparing the alpha6 and alpha6p integrin proteins. Repeated domains (shaded rectangles) are indicated by Roman numerals I-VII. The putative ligand- and cation-binding domains are contained between repeated domains III and IV and domains V and VI, respectively. Exons 1-25 of the alpha6 integrin sequence are indicated. The mapped sequence positions of two anti-alpha6A antibodies (AA6A and 4E9G8) that recognize both alpha6 and the alpha6p variant are shown by an asterisk on the full-length alpha6 schematic. Conformationally dependent epitopes for anti-alpha6 integrin antibodies used for immunoprecipitation are not indicated on the schematic.

FIG. 8 is an image of a Western blot (A) of alpha6 protein immunoprecipitated from surface-biotinylated DU145H cells, and a graph (B) of densitometry values illustrating the decay rates of alpha6, beta1 and alpha6p. DU145H cells were surface-biotinylated and incubated for 24, 48, or 72 h, followed by lysis and immunoprecipitation with anti-alpha6 antibody GoH3. The samples were analyzed by a non-reducing 7.5% polyacrylamide gel, transferred to polyvinylidene fluoride membrane, reacted with peroxidase-conjugated streptavidin, and visualized by chemiluminescence (A). The asterisk indicates a biotinylated protein band that was variably seen and is of unknown identity. The film was digitized, and the densitometry values were analyzed for relative degradation rates of alpha6, beta1, and alpha6p. Decay rates are illustrated in the graph in panel B.

FIG. 9 is an image of a 1× TBE-1.5% agarose gel showing an RT-PCR amplification of the alpha6 integrin coding region and subsequent diagnostic digests. PCR primers that bracketed the integrin alpha6 coding region were used to amplify first strand cDNA generated from RNA isolated from DU145H cells (lane 1). To confirm the identity of the integrin alpha6 PCR product, aliquots were digested with four diagnostic restriction enzymes (EcoN I, lane 2; EcoR I, lane 3; Sma I, lane 4; and Xho I, lane 5), size separated, and visualized by ethidium bromide staining. The molecular mass standard is EcoR I/Hind III-digested DNA (lane M).

FIG. 10 is an image of a Western blot (A) and graph (B) showing that calcium-induced normal keratinocyte differentiation increased alpha6p levels. The presence of alpha6 and alpha6p integrins was determined in normal 291 mouse keratinocytes, immortalized O3C nontumorigenic, and O3R tumorigenic derivatives. The cells were maintained in 0.4 mM calcium (low, lanes L) and switched to 0.14 mM (medium, lanes M) or 1.4 mM (high, lanes H) calcium medium at 60% confluency for 24 h of treatment, then frozen in a dry ice bath, and stored at 80° C. until analysis. Whole cell lysates (20 μg) were electrophoresed under non-reducing conditions on a 7.5% polyacrylamide gel and transferred to polyvinylidene fluoride membrane followed by Western blot analysis using anti-alpha6 integrin antibody, AA6A (A). The alpha6 and alpha6p integrin protein bands were scanned and quantitated using Scion Image and the results were graphed (B).

### DETAILED DESCRIPTION

The invention provides, in part, an isolated polypeptide that includes a truncated alpha6 integrin, also called an alpha6 integrin variant. In one embodiment, the N-terminal amino acid of the polypeptide sequence begins with an amino acid within exon 12 or 13 of the alpha6 integrin sequence, or within amino acids 517-1073, 571-1073, 588-1073, or 596-1073 of SEQ ID NO:1. The polypeptides of the invention can be protease stable and naturally occurring, and the polypeptides can be encoded by nucleotide sequences that are at least 80% identical to a corresponding region of the alpha6 cDNA (SEQ ID NO:2) or genomic sequences. Other exemplary alpha6 polypeptides can be encoded by sequences that hybridize to SEQ ID NO:2 or that include naturally-occurring variations, e.g., a variation described in GenBank® NM_{—}000210, e.g., a polymorphism at position 3804, 4120, 5120, 5338, or 5527, or in AH008066. The invention also provides an isolated nucleic acid that encodes a polypeptide that can be an N-terminally truncated alpha6 integrin variant, such as a protease-resistant alpha6 integrin variant, having an N-terminal amino acid that falls within exon 12 or 13 of the alpha6 integrin protein sequence. An exemplary polypeptide of the invention is the alpha6 integrin variant—alpha6p.

A polypeptide of the invention, including an isolated alpha6p, is a truncated form of the alpha6 integrin, wherein the truncation occurs at the N terminus. The novel N terminus can have an N-terminal amino acid that is located with exon 12, 13, or 14 of the alpha6 integrin. In addition, the novel polypeptide of the invention can have a light chain identical to the alpha6 integrin and may bind to either one or both of beta4 and beta1 integrin subunits. In some embodiments, it is possible to add a non-integrin sequence to the N-terminus of the truncated alpha6 integrin.

Alpha6p integrin, for example, is a naturally occurring variant that may be derived from proteolytic cleavage of alpha6. Such a cleavage event exposes a novel N-terminus in alpha6p. Alpha6p and alpha6 integrin have identical light chains, and alpha6b can bind to either the beta4 or beta1 subunits. Tryptic peptide sequencing of the alpha6p protein revealed peptide fragments having sequences identical to alpha6 integrin protein sequences encoded by exons 12-25 of the alpha6 integrin gene (Davis et al., Jour. Biol. Chem. 276:26099-26106, 2001; GenBank Accession No. AH008066). The amino acid sequence of human alpha6 integrin is known (see Tamura et al., J. Cell Biol. 111:1593-1604, 1990; GenBank Accession No. NP_{—}000201). An exemplary human alpha7 integrin amino acid sequence is shown as SEQ ID NO:1, as follows.

| | |
|---|---|
| MAAAGQLCLL YLSAGLLSRL GAAFNLDTRE DNVIRKYGDP GSLFGFSLAM HWQLQPEDKR | (SEQ ID NO:1) |
| LLLVGAPRGE ALPLQRANRT GGLYSCDITA RGPCTRIEFD NDADPTSESK EDQWMGVTVQ | |
| SQGPGGKVVT CAHRYEKRQH VNTKQESRDI FGRCYVLSQN LRIEDDMDGG DWSFCDGRLR | |
| GHEKFGSCQQ GVAATFTKDF HYIVFGAPGT YNWKGIVRVE QKNNTFFDMN IFEDGPYEVG | |
| GETEHDESLV PVPANSYLGF SLDSGKGIVS KDEITFVSGA PRANHSGAVV LLKRDMKSAH | |
| LLPEHIFDGE GLASSFGYDV AVVDLNKDGW QDIVIGAPQY FDRDGEVGGA VYVYMNQQGR | |
| WNNVKPIRLN GTKDSMFGIA VKNIGDINQD GYPDIAVGAP YDDLGKVFIY HGSANGINTK | |
| PTQVLKGISP YFGYSIAGNM DLDRNSYPDV AVGSLSDSVT IFRSRPVINI QKTITVTPNR | |
| IDLRQKTACG APSGICLQVK SCFEYTANPA GYNPSISIVG TLEAEKERRK SGLSSRVQFR | |
| NQGSEPKYTQ ELTLKRQKQK VCMEETLWLQ DNIRDKLRPI PITASVEIQE PSSRRRVNSL | |
| PEVLPILNSD EPKTAHIDVH FLKEGCGDDN VCNSNLKLEY KFCTREGNQD KFSYLPIQKG | |
| VPELVLKDQK DIALEITVTN SPSNPRNPTK DGDDAHEAKL IATFPDTLTY SAYRELRAFP | |
| EKQLSCVANQ NGSQADCELG NPFKRNSNVT FYLVLSTTEV TFDTPYLDIN LKLETTSNQD | |
| NLAPITAKAK VVIELLLSVS GVAKPSQVYF GGTVVGEQAM KSEDEVGSLI EYEFRVINLG | |
| KPLTNLGTAT LNIQWPKEIS NGKWLLYLVK VESKGLEKVT CEPQKEINSL NLTESHNSRK | |
| KREITEKQID DNRKFSLFAE RKYQTLNCSV NVNCVNIRCP LRGLDSKASL ILRSRLWNST | |
| FLEEYSKLNY LDILMRAFID VTAAAENIRL PNAGTQVRVT VFPSKTVAQY SGVPWWIILV | |
| AILAGILMLA LLVFILWKCG FFKRNKKDHY DATYHKAEIH AQPSDKERLT SDA | |

Exemplary variants include G69A, P289G, F501L, and D805Y.

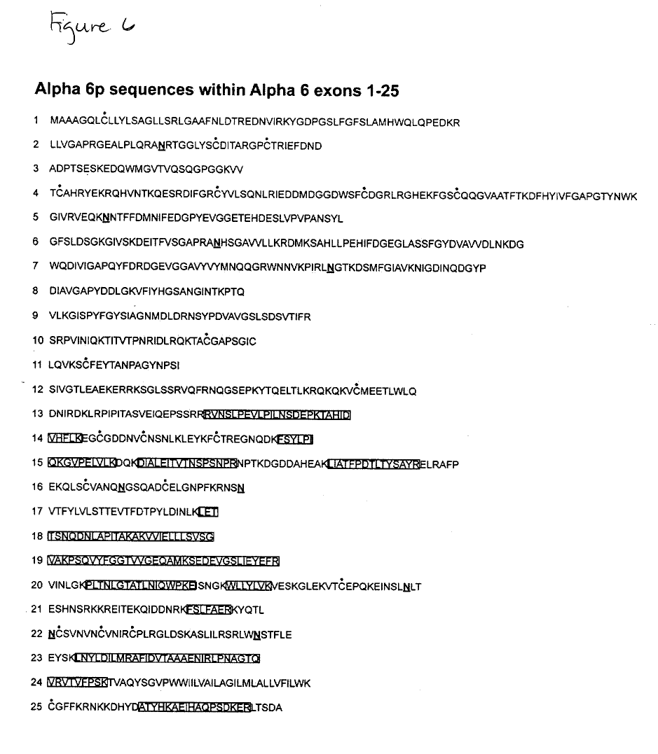

The amino acid sequences of alpha6p peptide fragments were identified by peptide sequencing. The most N terminal fragment began with the sequence RVN, at amino acids 596-598 of SEQ ID NO:1 (see FIG. 6). In addition, because alpha6p is a C-terminal fragment of alpha6, alpha6p does not contain N-terminal epitopes of alpha6 integrin, and accordingly does not bind to antibodies that recognize an N-terminal epitope of alpha6, such as A33 (Sterk et al., J. Cell Biol. 149:969-982, 2000).

A truncated alpha6 polypeptide of the invention can be protease-resistant. A “protease-resistant” peptide, as referenced herein, is as resistant or more resistant to degradation by proteases as alpha6 integrin. For example, a polypeptide of the invention will be resistant to intracellular and extracellular proteases in cell culture in vivo, when the polypeptide is presented on the surface of a cell (see Example 8, for example). An alpha6 variant of the invention can also be stable in the presence of exogenous protease inhibitors, such as BB94, leupeptin, aprotinin, 30% fetal bovine serum, or ecotin; exogenous proteases, such as kallikrein or plasmin; or activators of integrin function, such as 12-O-tetradecanoylphorbol-13-acetate (TPA) or phorbol 12-myristate 13-acetate (PMA). Stability of an alpha6 integrin variant of the invention can be assayed by a surface-biotinylation method. The surface half-life of the polypeptide is determined by biotinylating the surface proteins of a cell expressing the polypeptide. Following biotinylation for 10, 20, 40 min, 1 hr, 2 hr, 4 hr or longer, preferably 0.5-1.5 hr, the cells can be washed and placed back in an incubator in fresh medium. The integrin variant can be immunoprecipitated, or otherwise isolated, from the cell culture at increasing time points, depending on the rate of cell growth and polypeptide stability. For a human cell, for example, at least one time point can be taken every 12 hours or every 24 hours or every 48 hours for 2, 3, 4, 5 days or more, preferably for at least 3 days. The appropriate time point can be influenced by cell type and can be determined empirically. The quantity of biotinylated integrin variant can be determined by chemiluminescence, such as on a Western blot. The rate of decline of biotinylation signal over a period of time is indicative of the rate at which the integrin variant is cleared from the surface of the cells.

The invention also provides an isolated nucleic acid sequence that encodes an alpha6 variant polypeptide having one or more of the properties described above. The polypeptide is an N-terminal truncation of alpha6, and the N terminal amino acid sequence of the variant polypeptide is within exon 12, 13, or 14 of alpha6 integrin, and preferably within amino acids 517-596 of SEQ ID NO:1. The polypeptide can also be protease-resistant and a naturally occurring variant. The nucleic acid can include a regulatory sequence, e.g., a 3′ UTR. For example, exons 12-26 of the alpha6 integrin include the C-terminus of the gene and the 3′ untranslated region (UTR), which is located in sequences spanning exons 25 and exon 26.

The nucleic acids of the invention can include vector nucleic acids. The vector can be a plasmid, such as an expression vector, or a viral vector or yeast expression vector.

The invention also provides for a host cell, which contains a nucleic acid that encodes an N-terminal truncated alpha6 integrin variant, but does not include a sequence coding for the N-terminal region of alpha6 integrin, e.g., which does include nucleic acid sequences corresponding to exon 1-7, or exons 1-11. The host cell can be a mammalian cell, such as a mouse or human cell. Exemplary mouse cell lines include 291, O3C and O3R cells. Exemplary human cell lines of the invention include DU145H (prostate carcinoma), HaCaT (normal immortalized keratinocyte), PC3-N (prostate tumor), MCF-7 (breast tumor), PC3-ATCC (prostate tumor), LnCap (prostate carcinoma), H69, SW480 (colon carcinoma), PrEC (normal prostate cells), and PC3 (prostate tumor).

Producing a Truncated Alpha 6 Integrin.

The invention also provides for a method of producing an N-terminal truncated alpha6 variant peptide. The method includes culturing a host cell that contains a nucleic acid encoding the polypeptide under the appropriate conditions. For example, a mouse cell, such as from a 291, O3C or O3R cell line, can be maintained in calcium conditions ranging from 0.01 to 2.0 mM, preferably 0.04-1.4 mM. For a mouse 291 cell in particular, terminal differentiation induced by high calcium levels (0.8-2.0 mM, preferably 1.0-1.5 mM, more preferably 1.4 mM) can increase production of an alpha6 integrin variant polypeptide, particularly an alpha6p polypeptide.

A human cell can also be cultured to produce an alpha6 integrin variant polypeptide. A human cell line can be cultured, for example, at 37° C. in a humidified atmosphere of 95% air and 5% CO₂. Human cells can be cultured in medium, such as Iscove's modified Dulbecco's medium (Invitrogen Corporation, Carlsbad, Calif.; formerly Life Technologies, Inc.) plus 10% fetal bovine serum; Dulbecco's modified Eagle's medium (Invitrogen Corporation, Carlsbad, Calif.; formerly Life Technologies, Inc.) plus 10% fetal bovine serum; Dulbecco's modified Eagle's medium plus 5% nonessential amino acids, 5% L-glutamine, 5% sodium pyruvate, 10% fetal bovine serum; or PrEGM bullet kit medium (Clonetics, San Diego, Calif.). Optimum culture conditions can depend on the type, e.g., origin, of the cell line and can be determined empirically.

Standard recombinant nucleic acid methods can be used to express a protein that includes a truncated alpha6 integrin. Generally, a nucleic acid sequence encoding the protein is cloned into a nucleic acid expression vector. In a typical embodiment, the nucleic acid does not include sequences encoding amino acids sequence segments of the full length alpha6 integrin that are absent from the truncated alpha6 integrin. Information about the genetic code can be used to design an appropriate coding nucleic acid, e.g., by fusion to a signal sequence so that cleavage of the signal sequence will produce the truncated alpha6 integrin (e.g., at least the extracellular domain) in a secreted or transmembrane form. The expression vector for expressing the protein can include, in addition to the segment encoding the protein or fragment thereof, regulatory sequences, including for example, a promoter, operably linked to the nucleic acid(s) of interest. Large numbers of suitable vectors and promoters are known to those of skill in the art and are commercially available for generating the recombinant constructs of the present invention. The following vectors are provided by way of example. Bacterial: pBs, phagescript, PsiX174, pBluescript SK, pBs KS, pNH8a, pNH16a, pNH18a, pNH46a (Stratagene); pTrc99A, pKK223-3, pKK233-3, pDR540, and pRIT5 (Pharmacia). Eukaryotic: pWLneo, pSV2cat, pOG44, PXTI, pSG (Stratagene) pSVK3, pBPV, pMSG, and pSVL (Pharmacia).

Methods well known to those skilled in the art can be used to construct vectors containing a polynucleotide of the invention and appropriate transcriptional/translational control signals. These methods include in vitro recombinant DNA techniques, synthetic techniques and in vivo recombination/genetic recombination. See, for example, the techniques described in Sambrook & Russell, Molecular Cloning: A Laboratory Manual, 3^{rd} Edition, Cold Spring Harbor Laboratory, N.Y. (2001) and Ausubel et al., Current Protocols in Molecular Biology (Greene Publishing Associates and Wiley Interscience, N.Y. (1989). Promoter regions can be selected from any desired gene using CAT (chloramphenicol transferase) vectors or other vectors with selectable markers. Two appropriate vectors are pKK232-8 and pCM7. Particular named bacterial promoters include lacI, lacZ, T3, T7, gpt, lambda P, and trc. Eukaryotic promoters include CMV immediate early, HSV thymidine kinase, early and late SV40, LTRs from retrovirus, mouse metallothionein-I, and various art-known tissue specific promoters.

Generally, recombinant expression vectors will include origins of replication and selectable markers permitting transformation of the host cell, e.g., the ampicillin resistance gene of E. coli and S. cerevisiae auxotrophic markers (such as URA3, LEU2, HIS3, and TRPl genes), and a promoter derived from a highly expressed gene to direct transcription of a downstream structural sequence. Such promoters can be derived from any gene, for example, from operons encoding glycolytic enzymes such as 3-phosphoglycerate kinase (PGK), a-factor, acid phosphatase, or heat shock proteins, among others, or it can be a synthetic promoter. The polynucleotide of the invention is assembled in appropriate phase with translation initiation and termination sequences, and preferably, a leader sequence capable of directing secretion of translated protein into the periplasmic space or extracellular medium. Optionally, a nucleic acid of the invention can encode a fusion protein including an N-terminal identification peptide imparting desired characteristics, e.g., stabilization or simplified purification of expressed recombinant product. Useful expression-vectors for bacteria are constructed by inserting a polynucleotide of the invention together with suitable translation initiation and termination signals, optionally in operable reading phase with a functional promoter. The vector will comprise one or more phenotypic selectable markers and an origin of replication to ensure maintenance of the vector and to, if desirable, provide amplification within the host. Suitable prokaryotic hosts for transformation include E. coli, Bacillus subtilis, Salmonella typhimurium and various species within the genera Pseudomonas, Streptomyces, and Staphylococcus, although others may also be employed as a matter of choice.

As a representative but nonlimiting example, useful expression vectors for bacteria can comprise a selectable marker and bacterial origin of replication derived from commercially available plasmids comprising genetic elements of the well known cloning vector pBR322 (ATCC 37017). Such commercial vectors include, for example, pKK223-3 (Pharmacia Fine Chemicals, Uppsala, Sweden) and pGEM1 (Promega, Madison, Wis., USA).

The present invention further provides host cells containing the vectors of the present invention, wherein the nucleic acid has been introduced into the host cell using known transformation, transfection or infection methods. The host cell can be a eukaryotic host cell, such as a mammalian cell, a lower eukaryotic host cell, such as a yeast cell, or the host cell can be a prokaryotic cell, such as a bacterial cell. Introduction of the recombinant construct into the host cell can be effected, for example, by calcium phosphate transfection, DEAE, dextran mediated transfection, or electroporation (Davis, L. et al., Basic Methods in Molecular Biology (1986)).

Any host/vector system can be used to identify one or more of the target elements of the present invention. These include, but are not limited to, eukaryotic hosts such as HeLa cells, CV-1 cell, COS cells, and Sf9 cells, as well as prokaryotic host such as E. coli and B. subtilis. The most preferred cells are those which do not normally express the particular reporter polypeptide or protein or which express the reporter polypeptide or protein at low natural level.

The host of the present invention may also be a yeast or other fungi. In yeast, a number of vectors containing constitutive or inducible promoters may be used. For a review see, Current Protocols in Molecular Biology, Vol. 2, Ed. Ausubel et al., Greene Publish. Assoc. & Wiley Interscience, Ch. 13 (1988); Grant et al., Expression and Secretion Vectors for Yeast, in Methods in Enzymology, Ed. Wu & Grossman, Acad. Press, N.Y. 153:516-544 (1987); Glover, DNA Cloning, Vol. II, IRL Press, Wash., D.C., Ch. 3 (1986); Bitter, Heterologous Gene Expression in Yeast, in Methods in Enzymology, Eds. Berger & Kimmel, Acad. Press, N.Y. 152:673-684 (1987); and The Molecular Biology of the Yeast Saccharomyces, Eds. Strathern et al., Cold Spring Harbor Press, Vols. I and 11 (1982).

The host of the invention may also be a prokaryotic cell such as E. coli, other enterobacteriaceae such as Serratia marescans, bacilli, various pseudomonads, or other prokaryotes which can be transformed, transfected, infected.

The present invention further provides host cells genetically engineered to contain the polynucleotides that encode a protein that includes a truncated alpha6 integrin. For example, such host cells may contain nucleic acids of the invention introduced into the host cell using known transformation, transfection or infection methods. The present invention still further provides host cells genetically engineered to express the polynucleotides, wherein such polynucleotides are in operative association with a regulatory sequence heterologous to the host cell which drives expression of the polynucleotides in the cell.

The host cell can be a higher eukaryotic host cell, such as a mammalian cell, a lower eukaryotic host cell, such as a yeast cell, or the host cell can be a prokaryotic cell, such as a bacterial cell.

Introduction of the recombinant construct into the host cell can be effected by calcium phosphate transfection, DEAE, dextran mediated transfection, or electroporation (Davis, L. et al., Basic Methods in Molecular Biology (1986)). The host cells containing one of polynucleotides of the invention, can be used in conventional manners to produce the gene product encoded by the isolated fragment (in the case of an ORF).

Any host/vector system can be used to express one or more of the diversity strands of the present invention. These include, but are not limited to, eukaryotic hosts such as HeLa cells, CV-1 cells, COS cells, and Sf9 cells, as well as prokaryotic host such as E. coli and B. subtilis. The most preferred cells are those which do not normally express the particular polypeptide or protein or which express the polypeptide or protein at low natural level. Mature proteins can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook, et al., in Molecular Cloning: A Laboratory Manual, Second Edition, Cold Spring Harbor, N.Y. (1989), the disclosure of which is hereby incorporated by reference.

Various mammalian cell culture systems can also be employed to express recombinant protein.

Examples of mammalian expression systems include the COS-7 lines of monkey kidney fibroblasts, described by Gluzman, Cell 23:175 (1981), and other cell lines capable of expressing a compatible vector, for example, the C127, 3T3, CHO, HeLa and BHK cell lines. Mammalian expression vectors will comprise an origin of replication, a suitable promoter and also any necessary ribosome-binding sites, polyadenylation site, splice donor and acceptor sites, transcriptional termination sequences, and 5′ flanking nontranscribed sequences.

DNA sequences derived from the SV40 viral genome, for example, SV40 origin, early promoter, enhancer, splice, and polyadenylation sites may be used to provide the required nontranscribed genetic elements. Recombinant polypeptides and proteins produced in bacterial culture are usually isolated by initial extraction from cell pellets, followed by one or more salting-out, aqueous ion exchange or size exclusion chromatography steps. In some embodiments, the template nucleic acid also encodes a polypeptide tag, e.g., penta- or hexahistidine. The recombinant polypeptides encoded by a library of diversity strands can then be purified using affinity chromatography.

Microbial cells employed in expression of proteins can be disrupted by any convenient method, including freeze-thaw cycling, sonication, mechanical disruption, or use of cell lysing agents. A number of types of cells may act as suitable host cells for expression of the protein. Scopes (1994) Protein Purification: Principles and Practice, New York:Springer-Verlag provides a number of general methods for purifying recombinant (and non-recombinant) proteins. The method include, e.g., ion-exchange chromatography, size-exclusion chromatography, affinity chromatography, selective precipitation, dialysis, and hydrophobic interaction chromatography.

Mammalian host cells include, for example, monkey COS cells, Chinese Hamster Ovary (CHO) cells, human kidney 293 cells, human epidermal A431 cells, human Colo205 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK or Jurkat cells.

Alternatively, it may be possible to produce the protein in lower eukaryotes such as yeast or in prokaryotes such as bacteria. Potentially suitable yeast strains include Saccharomyces cerevisiae, Schizosaccharomyces pombe, Kluyveromyces strains, Candida, or any yeast strain capable of expressing heterologous proteins. Potentially suitable bacterial strains include Escherichia coli, Bacillus subtilis, Salmonella typhimurium, or any bacterial strain capable of expressing heterologous proteins. If the protein is made in yeast or bacteria, it may be necessary to modify the protein produced therein, for example by phosphorylation or glycosylation of the appropriate sites, in order to obtain the functional protein. Such covalent attachments may be accomplished using known chemical or enzymatic methods. In another embodiment of the present invention, cells and tissues may be engineered to express an endogenous gene comprising the polynucleotides of the invention under the control of inducible regulatory elements, in which case the regulatory sequences of the endogenous gene may be replaced by homologous recombination. As described herein, gene targeting can be used to replace a gene's existing regulatory region with a regulatory sequence isolated from a different gene or a novel regulatory sequence synthesized by genetic engineering methods.

Such regulatory sequences may be comprised of promoters, enhancers, scaffold-attachment regions, negative regulatory elements, transcriptional initiation sites, regulatory protein binding sites or combinations of said sequences. Alternatively, sequences which affect the structure or stability of the RNA or protein produced may be replaced, removed, added, or otherwise modified by targeting, including polyadenylation signals. mRNA stability elements, splice sites, leader sequences for enhancing or modifying transport or secretion properties of the protein, or other sequences which alter or improve the function or stability of protein or RNA molecules.

Evaluating a Sample for a Disease or Disorder

Various disease states involving epithelial cells have been associated with alterations in alpha6 integrin-containing heterodimers. Mice completely lacking the alpha6 integrin will develop to birth but die shortly thereafter because of severe blistering of the skin and other epithelia (Georges-Labouesse et al., Nat. Genet. 13:370-373, 1996). Alterations in the alpha6 integrin and/or a deficiency of its pairing subunit, beta4 integrin, are associated with pyloric atresia-junctional epidermolysis bullosa, a human blistering disease of the epithelia (Brown et al., J. Invest. Dermatol. 107:384-391, 1996; Shimizu et al., Arch. Dermatol. 132:919-925, 1996; Vidal et al., Nat. Genet. 10:229-234, 1995; Pulkkinen et al., Lab. Invest. 76:823-833, 1997; Niessen et al., J. Cell Sci. 109:1695-1706, 1996; Gil et al., J. Invest. Dermatol. 103:31S-38S, 1994).

Investigation of a human epithelial cancer indicated a deficiency of the alpha6beta4 heterodimer pairing during prostate tumor progression (Cress et al., Cancer Metastasis Rev. 14:219-228, 1995; Nagle et al., Am. J. Pathol. 146:1498-1507, 1995) and a persistent expression of the alpha6beta1 integrin (Knox et al., Am. J. Pathol. 145:167-174, 1994). Other studies have revealed the persistent nonpolarized expression of the alpha6 integrin during human tumor progression in cancers arising within the breast (Friedrichs et al., Cancer Res. 55:901-906, 1995), kidney (Droz et al., Lab. Invest. 71:710-718, 1994), endometrium (Lessey et al., Am. J. Pathol. 146:717-726, 1995), and pancreas (Weinel et al., Int. J. Cancer 52:827-833, 1992; Weinel et al., Gastroenterology 108:523-532, 1995), in addition to micrometastases from solid epithelial tumors (Putz et al., Cancer Res. 59:241-248, 1999). Immunoprecipitation of alpha6 integrin from human prostate cancer cells using alpha6-specific monoclonal antibodies retrieved not only the expected alpha1 and beta4 subunits but also a predominant protein with an apparent molecular mass of 70 kDa (Witkowski et al., J. Cancer Res. Clin. Oncol. 119:637-644, 1993; Rabinovitz et al., Clin. Exp. Metastasis 13:481-491, 1995). The experiments presented below (see Examples) show that the protein is a novel and smaller form of the alpha6 integrin that is capable of pairing with either the alpha1 or beta4 integrin subunit. The integrin is referred to herein as an N-terminally truncated alpha6 integrin, and is called alpha6p for the latin word parvus, meaning small.

The invention provides a method for evaluating a sample for a proliferative disorder or epithelial disorder, such as a cancer or a blistering disorder. The method can include detecting an N-terminally truncated alpha6 integrin variant in the cell or tissue using an antibody described herein, and thereby determining that the cell or tissue has a proliferative disorder or is at risk for a proliferative disorder, or if an epithelial tissue has an epithelial-specific disorder or is at risk for an epithelial disorder. The sample can be obtained from a mammal, such as a human or a mouse. The cell can be obtained from a tissue of the body, such as the prostate, breast, colon, lung, skin or other epithelial tissue. Failure to detect the alpha6 integrin variant may not indicate the absence of a proliferative disorder. The presence of the variant polypeptide can be detected by immunoprecipitation and/or a Western blot analysis using antibodies described herein.

The invention is further illustrated by the following examples, which should not be construed as further limiting. The contents of all references, pending patent applications and published patents, cited throughout this application are hereby expressly incorporated by reference.

### EXAMPLES

A 70-kDa (nonreduced) form of the alpha6 integrin, called alpha6p for the Latin word parvus, meaning small, was identified and characterized as follows.

### Example 1

Materials and Methods

Cell Lines. All human cell lines were incubated at 37° C. in a humidified atmosphere of 95% air and 5% CO₂. Cell lines DU145H, HaCaT, and PC3-N were grown in Iscove's modified Dulbecco's medium (Invitrogen Corporation, Carlsbad, Calif.; formerly Life Technologies, Inc.) plus 10% fetal bovine serum. Cell lines MCF-7, PC3-ATCC, LnCap, and H69 were grown in Dulbecco's modified Eagle's medium (Invitrogen Corporation, Carlsbad, Calif.; formerly Life Technologies, Inc.) plus 10% fetal bovine serum. SW480 cells were grown in super medium (Dulbecco's modified Eagle's medium plus 5% nonessential amino acids, 5% L-glutamine, 5% sodium pyruvate, 10% fetal bovine serum). Normal prostate cells, PrEC, were grown in PrEGM bullet kit medium (Clonetics, San Diego, Calif.). The following cell lines were obtained from the American Type Culture Collection (Manassas, Va.): MCF-7 (human breast tumor), PC3 (human prostate tumor), LnCap (prostate carcinoma cell line), H69 (human lung carcinoma), and SW480 (human colon carcinoma). The DU145H cells were isolated as described previously (Rabinovitz et al., Clin. Exp. Metastasis 13:481-491, 1995) and contain only the alpha6A splice variant (Cress et al., Cancer Metastasis Rev. 14:219-228, 1995). The PC3-N cells are a variant of PC3 prostate carcinoma cell line (Tran et al., Am. J. Pathol. 155:787-798, 1999). The HaCaT cells (normal immortalized keratinocyte cell line) (Breitkreutz et al., Eur J. Cell Biol. 75:273-286, 1998) were obtained from Dr. Norbert E. Fusenig (German Cancer Research Center, University of Heidelberg, Heidelberg, Germany). PrEC (a normal prostate cell line) was obtained from Clonetics™ (Cambrex, East Rutherford N.J.). The calcium-induced terminal differentiation assay, cell culture techniques, and preparation of calcium medium used for mouse 291, 03C, and 03R cells have been described previously (Kulesz-Martin et al., Carcinogenesis 1:995-1006, 1980; Hennings et al., Curr. Probl. Dermatol. 10:3-25, 1980). Cells were maintained in 0.04 mM calcium (low calcium) and switched to medium with 0.14 mM calcium (medium calcium) or 1.4 mM calcium (high calcium) by 60% confluency. After 24 h treatment, cells were collected in phosphate-buffered saline, centrifuged, frozen in a dry ice bath, and kept at 80° C. in a freezer until used.

Antibodies. Anti-alpha6 integrin antibodies include and were obtained as follows. GoH3, a rat IgG2a, was from Accurate Chemicals (Westbury, N.Y.) (Sonnenberg et al., J. Biol. Chem. 262:10376-10383, 1987); J1B5, a rat monoclonal antibody, was obtained from Dr. Caroline Damsky (University of California, San Francisco, Calif.) (Damsky et al., Development 120:3657-3666, 1994); 4F10, a mouse IgG2b, was from Chemicon (Temcula, Calif.) (Sonnenberg et al., J. Biol. Chem. 262:10376-1038,1987); BQ16, a mouse IgG1 that recognizes an extracellular epitope of the alpha6 integrin, was obtained from Dr. Monica Leibert (Department of Urology, University of Texas, M.D. Anderson Cancer Center, Houston, Tex.) (Liebert et al., Hybridoma 12:67-80, 1993); 4E9G8, a mouse IgG1 that is specific for the unphosphorylated alpha6A cytoplasmic tail, was from Immunotech (Marseille, France) (Sonnenberg et al., Nature 336:487-489, 1988; Hemler et al., J. Biol. Chem. 263:7660-7665, 1988); AA6A, a rabbit polyclonal antibody that was raised and purified by Bethyl Laboratories Inc. (Montgomery, Tex.) specific for the last 16 amino acids (CIHAQPSDKERLTSDA; SEQ ID NO:8) of the human alpha6A sequence (Tamura et al., J. Cell Biol. 111:1593-1604, 1990) as done previously (Cooper et al., J. Cell Biol. 115:843-850, 1991), and A33, a rabbit polyclonal antibody that was raised against amino acids 1-500 of the alpha6 integrin (Sterk et al., J. Cell Biol. 149:969-982, 2000), from the Netherlands Cancer Institute. Anti-alpha4 integrin antibodies were obtained as follows. 3E1, a mouse ascites IgG1, was from Invitrogen Corporation (Carlsbad, Calif.; formerly Life Technologies, Inc.) (Hessle et al., Differentiation 26:49-54, 1984); 439.9b, a rat IgG2bK, was from Pharmingen (San Diego, Calif.) (Falcioni et al., Cancer Res. 48:816-821, 1988); ASC-3, a mouse IgG1K, was from Chemicon (Temecula, Calif.) (Pattaramalai et al., Exp. Cell Res. 222:281-290, 1996); and A9, a mouse IgG2a, was from Ancell (Bayport, Minn.) (Van Waes et al., Cancer Res. 51:2395-2402, 1991). Other anti-integrin antibodies include anti-alpha5 integrin antibody P1D6, a mouse IgG3 (Invitrogen Corporation, Carlsbad, Calif.; formerly Life Technologies, Inc.) (Wayner et al., J. Cell Biol. 107:1881-1891, 1988), and anti-alpha1 integrin P4C10, a mouse ascites IgG1 (Invitrogen Corporation, Carlsbad, Calif.; formerly Life Technologies, Inc.) (Carter et al., J. Cell Biol. 110:1387-1404, 1990).

Surface Biotinylation of Cell Lines. Previous published protocols (Isberg and Leong, Cell 60:861-871, 1990; Einheber et al., J. Cell Biol. 123:1223-1236, 1993) were slightly modified. Briefly, cells were grown to confluency in 100-mm tissue culture dishes and washed three times with HEPES buffer (20 mM HEPES, 130 mM NaCl, 5 mM KCl, 0.8 mM MgCl₂, 1.0 mM CaCl₂, pH 7.45). The cells were then incubated with 2 ml of HEPES buffer supplemented with sulfosuccinimidyl hexanoate-conjugated biotin (500 μg/ml; Pierce), which is impermeable to cell membranes (Staros, Biochemistry 21:3950-3, 1982), to label cell surface proteins for 30 min at 4° C. The cells were washed three times and lysed in cold radioimmune precipitation buffer plus protease inhibitors (phenylmethylsulfonyl fluoride, 2 mM; leupeptin and aprotinin, 1 μg/ml). The lysate was briefly sonicated on ice before centrifugation at 10,000 rpm for 10 min, and the supernatant was collected for immunoprecipitations.

Immunoprecipitations. For immunoprecipitations, 200 μg of total protein lysate was used for each reaction and incubated with 35 μl of protein G-Sepharose and 1 μg of antibody. The final volume of the lysate was adjusted to 500 μl with radioimmune precipitation buffer (150 mM NaCl, 50 mM Tris, 5 mM EDTA, 1% (v/v) Triton X-100, 1% (w/v) deoxycholate, 0.1% (w/v) SDS, pH 7.5). The mixture was rotated for 18 h at 4° C. After incubation, complexes were washed three times with cold radioimmune precipitation buffer and cluted in 2× non-reducing sample buffer. Samples were boiled for 5 min prior to loading onto a 7.5% polyacrylamide gel for analysis. The proteins resolved in the gel were electrotransferred to Millipore (Billerica, Mass.) Immobilon-P polyvinylidene fluoride membrane, incubated with either peroxidase-conjugated streptavidin or Western blotting antibodies plus secondary antibody conjugated to horseradish peroxidase, and visualized by chemiluminescence (ECL Western blotting detection system; Amersham Pharmacia Biotech, Piscataway, N.J.).

Two-dimensional Nonreduced/Reduced Gel Electrophoreses. Nonreduced/reduced two-dimensional electrophoresis was done as described (Parker et al., J. Biol. Chem. 268:7028-7035, 1993). The samples were incubated in 0.625 M Tris-HCl, pH 6.8, 10% glycerol, 10% SDS, and applied to SDS-polyacrylamide gel electrophoresis (7.5% acrylamide) without reduction. The excised lanes were incubated in reducing sample buffer for 15 min and horizontally loaded at the top of a second dimension slab gel (also 7.5% acrylamide). The proteins were electrotransferred to polyvinylidene fluoride membrane (Millipore, Billerica, Mass.), incubated with either peroxidase-conjugated streptavidin or Western blotting primary antibodies followed by secondary antibody conjugated to horseradish peroxidase, and visualized by chemiluminescence (ECL Western blotting detection system; Amersham Pharmacia Biotech, Piscataway, N.J.).

Amino Acid Sequencing by Matrix-assisted Laser Desorption Ionization Mass Spectrometry and Liquid Chromatography-Tandem Mass Spectrometry. Amino acid sequencing of alpha6p was performed using two different analytical core services. For analytical core service at Deutsches Krebsforschungszentrum (Heidelberg, Germany), the alpha6p protein was immunoprecipitated using J1B5, and the proteins were separated by SDS-polyacrylamide gel electrophoresis (7.5%, 3 mm). After staining with Coomassie Blue, the alpha6p bands were excised, cut into small pieces (1×1 mm), washed, dehydrated (twice for 30 min with H₂O, twice for 15 min with 50% acetonitrile, and once for 15 min with acetonitrile), and incubated with 0.5 μg of trypsin in 20 μl of digest buffer (40 mM NH₄HCO₃, pH 8.0) at 37° C. for 16 h. The supernatant was subsequently analyzed by matrix-assisted laser desorption ionization (MALDI) mass spectrometry (Deutsches Krebsforschungszentrum) using thin film preparation technique. Aliquots of 0.3 μl of a nitrocellulose containing saturated solution of alpha-cyano-4-hydroxycinnamic acid in acetone were deposited onto individual spots on the target. Subsequently, 0.8 μl of 10% formic acid and 0.4 μl of the digest sample were loaded on top of the thin film spots and allowed to dry slowly at ambient temperature. To remove salts from the digestion buffer, the spots were washed with 5% formic acid and with H₂O. Sequence analysis was performed on a Procise 494 protein sequencer using a standard program supplied by Applied Biosystems (Foster City, Calif.). The FastA database searching program of Pearson and Lipman (Pearson and Lipman, Proc. Natl. Acad. Sci. U.S.A. 85:2444-2448, 1988) was used for database searching.

For sequence analysis at the Proteomics Core of the Arizona Cancer Center and Southwest Environmental Health Sciences Center of the University of Arizona, the alpha6p protein was immunoprecipitated using J1B5 antibody, and proteins were separated by SDS-polyacrylamide gel electrophoresis (7.5%, 3 mm). After staining with Coomassie Blue, the alpha6p bands were excised, cut into small pieces (1×1 mm), and subjected to in-gel digestion using trypsin as described previously (Shevchenko et al., Anal. Chem. 68:850-858, 1996). The extracted peptides following digestion were analyzed by liquid chromatography-tandem mass spectrometry using a quadrupole ion trap Finnigan LCQ classic mass spectrometer equipped with a quaternary pump P4000 HPLC and a Finnigan electrospray ionization source (ThermoFinnigan, San Jose, Calif.). The peptides were eluted from a reverse-phase C18 micro-column (Vydac 250×1 mm, Hesperia, Calif.) with a gradient of 3-95% acetonitrile in 0.5% formic acid and 0.01% trifluoroacetic acid over 150 min at a flow rate of 15 μl/min. Tandem mass spectrometry spectra of the peptides were analyzed with the SEQUEST program (Turbo Sequest) to assign peptide sequence to the spectra (Eng et al., J. Am. Soc. Mass Spectrom. 5, 1994). SEQUEST analyses were performed against the nonredundant database.

RT-PCR Analysis. Total cellular RNA was isolated by guanidium isothiocyanate cell lysis and cesium chloride purification (Chirgwin et al., Biochemistry 18:5294-5299, 1979). RNA was quantitated from spectrophotometric absorbance measurements at 260 nm. First strand cDNA was synthesized in a 30 μl reaction comprised of 1× PCR buffer (10 mM Tris, pH 8.3; 50 mM KCl; 1.5 mM MgCl₂); 1 mM each dATP, dCTP, dGTP, and dTTP; 100 pmol random hexamer; 20 units RNAsin; 200 units SuperScript reverse transcriptase II (Invitrogen Corporation, Carlsbad, Calif.; formerly Life Technologies, Inc.), and 3 μg of total cellular RNA incubated at 42° C. for 60 min. The reaction was terminated by incubation at 99° C. for 10 min. Integrin alpha6-specific PCR was performed by adding 80 μl of amplification reaction buffer (1× PCR buffer, 25 pmol of integrin alpha6-specific primers, and 2.5 units of Taq DNA polymerase) to the cDNA reaction, followed by incubation at 94° C. for 5 min and then 40 cycles of 94° C. for 1 min, 60° C. for 3 min, and 72° C. for 10 min, with a final extension at 72° C. for 5 min and a quick chill to 4° C. The PCR primers were derived from the integrin alpha6 cDNA sequence reported by Tamura et al. (J. Cell Biol. 111:1593-1604, 1990) (GenBank accession number X53586); the upstream primer sequence was from nucleotides 160 to 179, and the downstream primer was from nucleotides 3404 to 3423. The PCR product identity was confirmed by diagnostic restriction enzyme digests and size separation of the products through a 1× TBE, 1.5% agarose gel. The products were visualized by ethidium bromide staining and UV fluorescence.

### Example 2

DU145H Cells Contained a Smaller Form of the Alpha6 Integrin

Previous studies showed that the anti-alpha6 antibody GoH3 can immunoprecipitate a surface-biotinylated 70-kDa (non-reduced) protein from DU145H cells in addition to the expected 185-, 140-, and 120-kDa (non-reduced) proteins corresponding to the alpha4, alpha6, and alpha1 integrins, respectively (Witkowski et al., J. Cancer Res. Clin. Oncol. 119:637-644, 1993; Rabinovitz et al., Clin. Exp. Metastasis 13:481-491, 1995). In DU145H cells, which only contain the alpha6A splice variant of alpha6 integrin (Cress et al., Cancer Metastasis Rev. 14:219-228, 1995), this 70-kDa variant was the predominant form of the alpha6 integrin found on the cell surface.

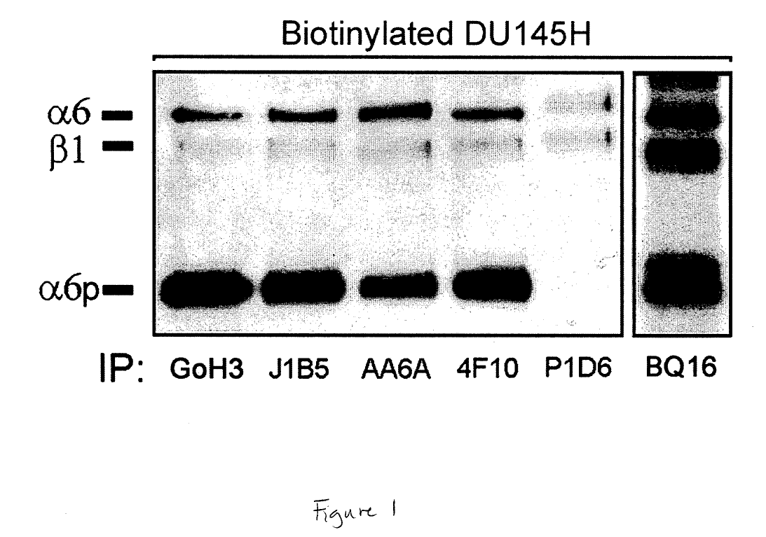

Five different anti-alpha6 antibodies immunoprecipitated alpha6 and its smaller variant, alpha6p, from surface-biotinylated DU145H cells (FIG. 1). Four of the antibodies used were specific for extracellular epitopes of the full-length alpha6A integrin (GoH3, J1B5, 4F10, and BQ16), and one was specific for the cytoplasmic tail of the alpha6A light chain (AA6A). The integrin alpha6p was not found to co-immunoprecipitate upon incubation with an anti-alpha3 antibody, P1B5 (data not shown), or an anti-alpha5 antibody, P1D6.

### Example 3

The Alpha6p Variant Contained a Light Chain that was Identical to That Found in Alpha6 Integrin

The full-length alpha6 integrin consists of two disulfide-linked chains, a heavy chain (110-kDa) and a cytoplasmic light chain (30-kDa) that are observed upon reduction of the protein samples and analysis by SDS-polyacrylamide gel electrophoresis. Western blot analysis (FIG. 1) indicated that an anti-alpha6 integrin antibody specific for the cytoplasmic tail of alpha6A (antibody AA6A) recognized alpha6p and suggested that the light chain from the alpha6p variant might be similar to that in the full-length alpha6 integrin.

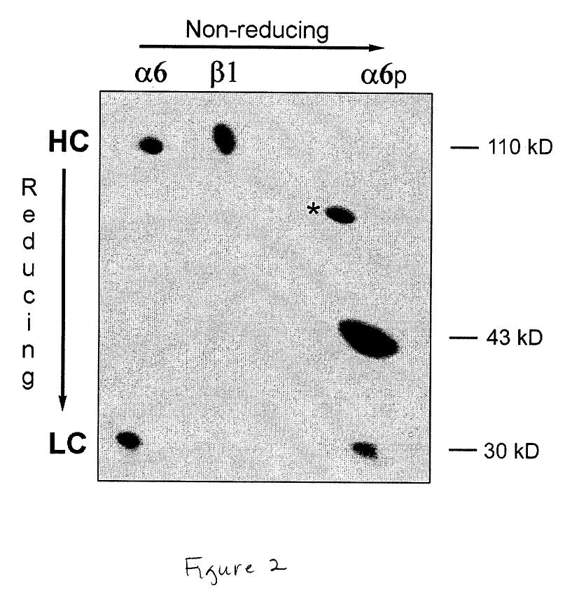

To test this hypothesis, DU145H cells were surface-biotinylated, and then immunoprecipitations were performed using the anti-alpha6 integrin antibody, GoH3. The immunoprecipitated protein was then analyzed using two-dimensional non-reducing/reducing gel electrophoresis (FIG. 2). The sample was electrophoresed under non-reducing conditions in the first dimension and then under reducing conditions for the second dimension. The 160-kDa band (non-reduced) corresponding to the full-length alpha6 integrin contained a heavy (110-kDa) and light (30-kDa) chain, as described previously (Hogervorst et al., Eur. J. Biochem. 199:425-433, 1991). The reduced alpha1 integrin was identified at 120-kDa. The alpha6p integrin split into a heavy fragment (43-kDa) and a light chain (30-kDa). These results indicated that the alpha6p integrin contained the same 30-kD light chain as the full-length alpha6A integrin but the heavy chains were significantly different.

### Example 4

The Alpha6p Variant Associated with Beta1 and Beta4 Integrins

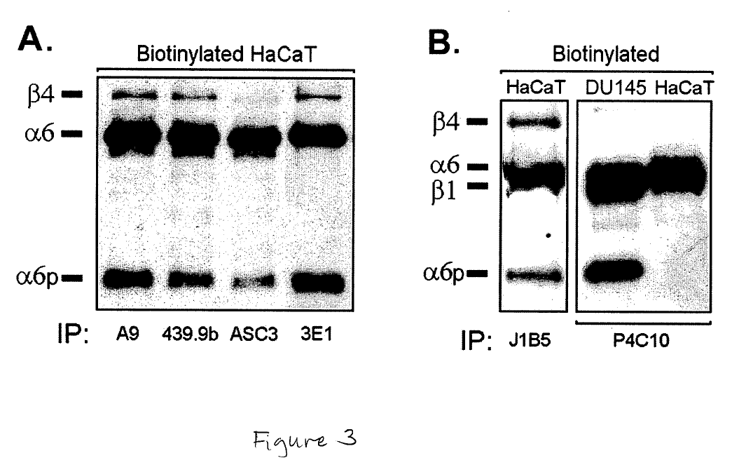

The alpha6 integrin is known to associate with either the beta1 or beta4 integrin subunit (Sonnenberg et al., J. Cell Sci. 96:207-217, 1990). Alpha6p was tested to determine whether it would co-immunoprecipitate with the beta4 integrin (FIG. 3A). Human HaCaT cells were chosen for this experiment because of their abundance of beta4 integrin (Witkowski et al., J. Cancer Res. Clin. Oncol. 119:637-644, 1993). The cells were surface-biotinylated and subjected to immunoprecipitation with different anti-beta4 integrin antibodies. The alpha6p variant co-immunoprecipitated upon incubation with four different anti-beta4 integrin antibodies: A9, 439.9b, ASC3, and 3E1. Of particular interest was the immunoprecipitation of alpha6p with the anti-beta4 integrin antibody, A9, whose epitope is present when alpha6 is coupled to beta4 integrin (Van Waes et al., Cancer Res. 51:2395-2402, 1991).

Next, the anti-beta1 integrin monoclonal antibody, P4C10, was tested for its ability to coimmunoprecipitate the novel 70-kDa (non-reduced) protein (FIG. 3B). HaCaT cells were surface-biotinylated and immunoprecipitated with anti-alpha6 integrin antibody, J1B5, and used as a standard. Both DU145 and HaCaT cells were surface-biotinylated and subjected to immunoprecipitation using P4C10. Interestingly, the 70-kDa (non-reduced) alpha6p variant co-immunoprecipitated with the beta1 integrin in DU145 cells but not in HaCaT cells. The results indicated that the novel alpha6p variant paired with either the beta4 or beta1 integrin subunits. Although the beta1 integrin was readily present in HaCaT cells, the alpha6p integrin did not co-immunoprecipitate with the anti-beta1 integrin antibody, P4C10.

### Example 5

The Alpha6p Integrin was Recognized by Light Chain-Specific Anti-Alpha6A Monoclonal Antibodies

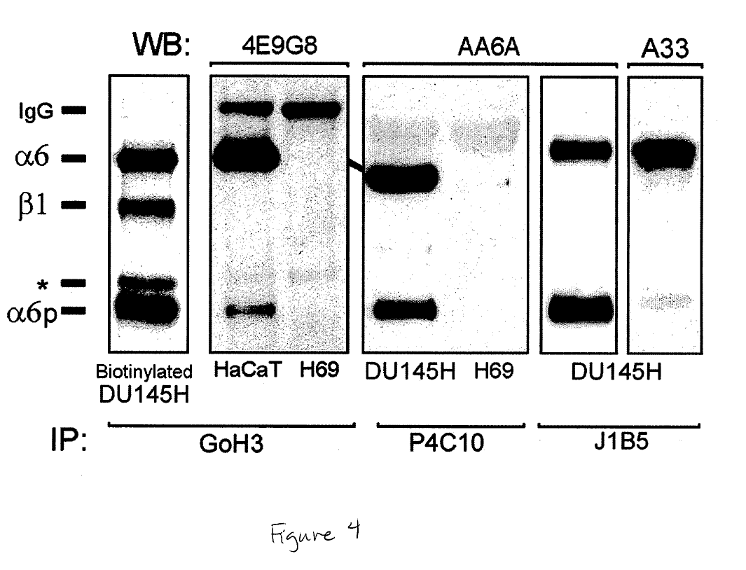

The data presented in (FIG. 2) indicated that alpha6p contained a light chain identical to that contained in the full-length alpha6 integrin. An experiment was performed to determine whether an anti-alpha6 integrin antibody could recognize the novel 70-kDa (non-reduced) protein by Western blot. DU145H cells were surface-biotinylated and immunoprecipitated with GoH3 antibodies for a standard to compare with a Western blot (FIG. 4). DU145H, HaCaT, and H69 cells were lysed and immunoprecipitated with either anti-alpha6 integrin antibodies GoH3 or J1B5 or anti-beta1 integrin monoclonal antibody, P4C10. A 70-kDa band that co-migrated with the biotinylated standard was recognized in HaCaT and DU145H cells by Western blot analysis using two different anti-alpha6A antibodies, AA6A and 4E9G8, which recognize the cytoplasmic domain of the alpha6A integrin. Additionally the alpha6 integrin, but not the alpha6p variant, was detected by Western blot analysis using A33, which is specific for the N-terminus of the alpha6 integrin. A lung carcinoma cell line, H69, is a cell line that does not contain alpha6 integrin and was not found to express alpha6p.

### Example 6

The Alpha6p Variant Was Present in Several Different Epithelial Cancer Cell Lines

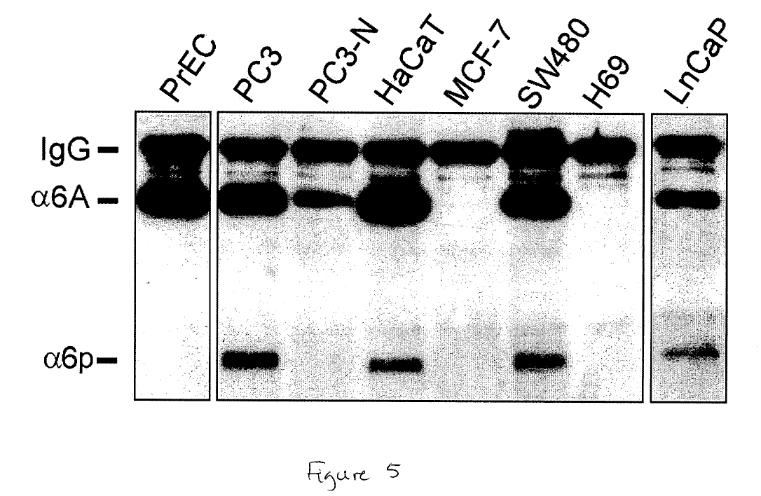

A variety of tumor and normal cell lines was examined for the presence of the alpha6p variant. The presence of alpha6 and alpha6p was initially analyzed by using whole cell lysates (20 μg of total protein) followed by Western blot analysis (data not shown). The results were tabulated and confirmed by immunoprecipitation with anti-alpha6 antibody GoH3 followed by Western blot analysis using anti-alpha6A antibody, AA6A (FIG. 5). The alpha6p variant was present in several prostate cancer cell lines (DU145H, PC3, and LnCaP) and a colon cancer cell line (SW480). Additionally, alpha6p was present in a normal, immortalized keratinocyte cell line, HaCaT. The alpha6p variant was not found in several cell lines including normal prostate cells, PrEC; a variant of the prostate cell line PC3, called PC3-N (Tran et al., Am. J. Pathol. 155:787-798, 1999); a breast carcinoma cell line, MCF-7; and a lung carcinoma cell line, H69. Interestingly, the alpha6p variant was only observed in cells that expressed the full-length alpha6 integrin. The alpha6p variant was not present in alpha6-negative cell lines. Two epithelial cell lines, one normal cell line (PrEC) and one cancer cell line (PC3-N), expressed the full-length alpha6 integrin but not the alpha6p variant.

### Example 7

The Alpha6p Variant Contained Several Amino Acid Fragments Identical to the Alpha6 Integrin

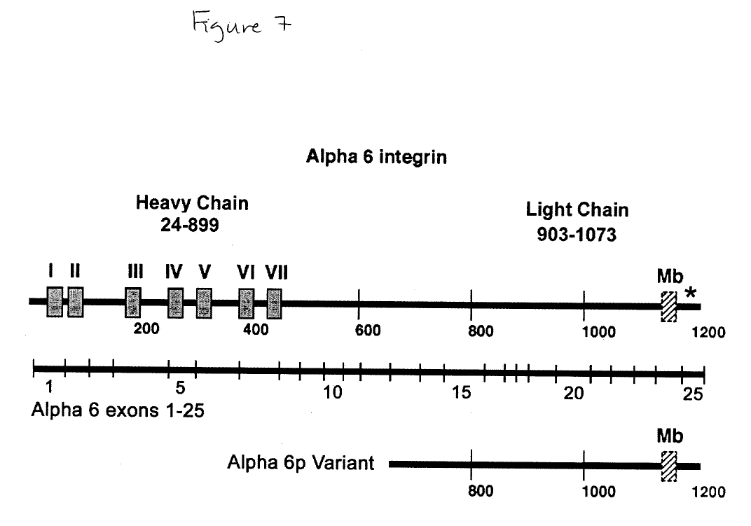

To determine more precisely the identity of the alpha6p variant, the protein was isolated and sequenced. To isolate the protein, alpha6p was immunoprecipitated with J1B5 antibody and electrophoresed. The protein gel was stained with Coomassie Blue, and then the 70-kDa protein was excised and digested with trypsin. Protein sequences were obtained using either MALDI mass spectrometry (Deutsches Krebsforschungszentrum) or liquid chromatography-tandem mass spectrometry (Proteomics Core of the Arizona Cancer Center and Southwest Environmental Health Sciences Center, University of Arizona) (FIG. 6). Ten noncontinuous amino acid fragments within the alpha6p variant were identified that corresponded exactly to predicted trypsin fragments located on exons 13-25 of the published alpha6 integrin sequence (Tamura et al, J. Cell Biol. 111:1593-1604, 1990). The sequencing data confirmed that both the heavy and light chains of the alpha6p variant contained identical portions of the full-length alpha6 integrin (FIG. 7).

### Example 8

The Alpha6p Variant Half-Life Was Three Times Longer than Alpha6

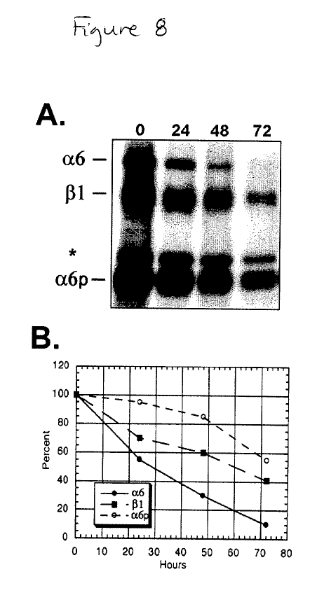

To determine if the novel alpha6p variant was a degradation product of the alpha6 integrin that would be rapidly cleared from the surface, the surface half-life of both integrins was examined using a biotinylation strategy. The surface proteins of DU145H cells were biotinylated for 1 h, washed, and placed back in the incubator with medium. After 24, 48, or 72 h, the integrins were immunoprecipitated using the GoH3 antibody and analyzed under non-reducing conditions (FIG. 8, panel A). The data indicated that the alpha6p form remained on the surface of the DU145H cells with a half-life of ˜72 h, or almost 3 times longer than that of the full-length alpha6 integrin (FIG. 8, panel B). The abundance of alpha6p was not influenced by exogenous protease inhibitors (BB94, leupeptin, aprotinin, 30% fetal bovine serum, ecotin), exogenous proteases (kallikrein), or activators of integrin function (12-O-tetradecanoylphorbol-13-acetate, 20 mM CaCl₂) (data not shown).

### Example 9

RT-PCR Analysis of the Alpha6 Coding Region Revealed a Single Product

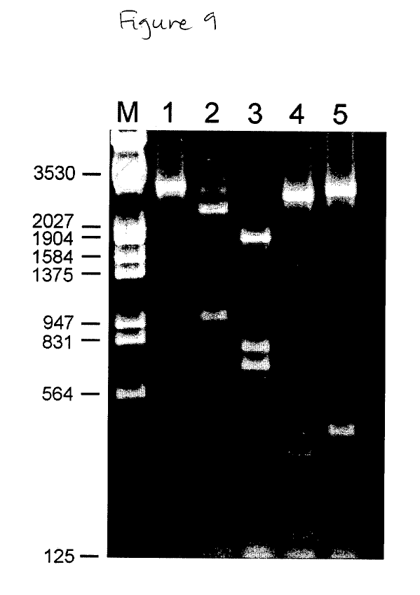

RT-PCR was used to determine whether splice variants of the integrin alpha6 mRNA could explain the production of the smaller integrin protein. Three micrograms of total cellular RNA from DU145H cells were reverse-transcribed into first strand cDNA and then PCR amplified with primers that bracketed most of the integrin alpha6 protein-coding region (all but the first four codons were amplified using these primers). The results of this experiment are shown in FIG. 9. A single PCR product consistent with a full-length RT-PCR product of 3263 bp was detected.

To confirm the identity of the integrin alpha6 PCR product, diagnostic restriction enzyme digests were performed. Analysis of the integrin alpha6 sequence (Tamura et al., J. Cell Biol. 111:1593-1604, 1990) revealed the presence of one EcoN I site (producing fragments of ˜960 and 2300 bp), four Sma I restriction sites (producing fragments of 105, 150, 350, and 2650 bp), four EcoR I sites (producing fragments of 30, 680, 730, and 1780 bp), and one Xho I site (producing fragments of 420 and 2840 bp). Aliquots of the integrin alpha6 PCR product were digested with each of these restriction enzymes, and the results of this experiment are shown in FIG. 9. Each restriction digest product produced the restriction fragments expected from the integrin alpha6 PCR product (the 30 bp EcoR I fragment and the 105 bp Sma I fragment could not be visualized on the gel shown in FIG. 9). Based on these results, it appears unlikely that the alpha6p variant is the result of the splicing out of exons in the known coding region.

### Example 10

Calcium-Induced Normal Keratinocyte Differentiation Increased Alpha6p Integrin Protein Levels

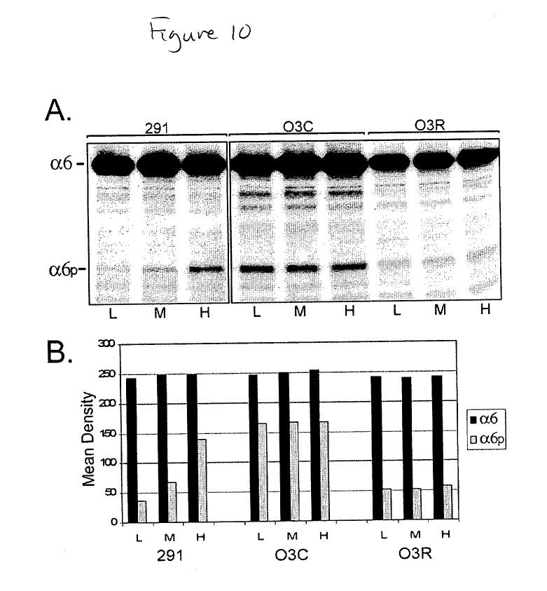

Mouse 291 normal keratinocyte terminal differentiation can be induced by calcium. O3C and O3R cells were derived from normal 291 mouse cell strains and are immortalized, nontumorigenic, and tumorigenic, respectively (Kulesz-Martin et al., Carcinogenesis 9:171-174, 1988). Both cell strains are resistant to calcium-induced terminal differentiation. The presence of alpha6 and alpha6p integrins in normal 291 mouse keratinocytes was determined using whole cell lysates followed by Western blot analysis using anti-alpha6 integrin antibody, AA6A (FIG. 10A). The results for 291 cells were confirmed by immunoprecipitation with anti-alpha6 integrin antibody, GoH3 (data not shown). The alpha6 and alpha6p integrin protein bands were quantitated using Scion Image (Cress, BioTechniques 29:776-781, 2000), and the results were graphed (FIG. 10B). Calcium-induced terminal differentiation increased alpha6p integrin protein levels 3-fold in a dose-dependent manner in 291 nontransformed mouse keratinocytes. The differing steady-state levels of alpha6p in proliferating O3C and O3R tumor cells under the same culture conditions suggested that the alpha6p integrin variant was responsive to terminal differentiation and not to calcium itself. Interestingly, alpha6p integrin levels were decreased in poorly differentiated squamous cell carcinoma O3R cells relative to initiated cell O3C precursors and terminally differentiated 291 keratinocytes.

### Example 11

Discussion

The alpha6 integrin is associated with an increased invasive potential of human prostate cancer cells in vitro and the progression of human prostate carcinoma in human tissue biopsy material. The alpha6 integrin exists in the classical form (140 kDa, nonreduced) and in a novel smaller form (70 kDa) referred to herein as alpha6p. The alpha6p is related to the full-length alpha6 because it was immunoprecipitated with anti-alpha6 integrin antibodies (GoH3, J1B5, AA6A, 4F10, and BQ16) (FIG. 1). Two-dimensional gel analysis revealed that the light chain of the alpha6p integrin was the same size as that found in the full-length alpha6 form (FIG. 2). The alpha6p variant co-immunoprecipitated with both anti-beta4 (3E1, A9, 439.9b, and ASC3) and anti-beta1 (P4C10) integrin antibodies (FIG. 3) and was recognized by two anti-alpha6 integrin antibodies specific for the cytoplasmic domain (AA6A and 4E9G8) by Western blot analysis but not by a polyclonal antibody that was specific for the N-terminal domain (A33) (FIG. 4). The alpha6p variant was found in several different human prostate (DU145H, LnCaP, and PC3) and colon (SW480) cancer cell lines (FIG. 5). It was not found in several cell lines including normal prostate cells (PrEC), a breast cancer cell line (MCF-7), a lung cancer cell line (H69), or a variant of a prostate carcinoma cell line (PC3-N). MALDI mass spectrometry indicated multiple amino acid regions in the alpha6p variant that corresponded exactly to sequences contained within exons 13-25 of the published full-length alpha6 sequence (Tamura, et al., J. Cell Biol. 111:1593-1604, 1990) (FIGS. 6 and 7). Calcium-induced terminal differentiation of normal mouse 291 keratinocytes resulted in a 3-fold increase of alpha6p protein levels (FIG. 10). Integrin modulation is known to occur in the differentiation of human keratinocytes (Watt and Jones, Dev. Suppl. 185-192, 1993). Modulation of calcium levels in 291 cell derivatives O3C and O3R cells that are both resistant to calcium-induced differentiation did not result in alterations of alpha6p integrin levels.

The ten noncontinuous amino acid fragments obtained from the alpha6p variant corresponded exactly to sequences contained within exons 13-25 of the full-length alpha6 integrin (FIGS. 6 and 7).

The predicted molecular mass of exons 13-25 is 55 kDa; yet the alpha6p protein band had an apparent molecular mass of 70 kDa by gel analysis. This apparent contradiction may be due to a post-translational modification of the protein. The full-length alpha6 integrin has a predicted molecular mass of 140 kDa (Tamura et al., J. Cell Biol. 111:1593-1604, 1990); yet experimentally, the protein band had an apparent molecular mass of 160 kDa under nonreducing conditions. The variation between predicted and apparent molecular mass in both proteins is likely due to the nine glycosylation sites predicted on the alpha6 protein and the five that would remain in alpha6p. Previously, differences in N-linked glycosylation of the alpha6 integrin, revealed by endoglycosidase H and N-glycanase treatments, has accounted for the variation in the apparent molecular mass of the alpha6 integrin from platelets and carcinoma lines (Sonnenberg et al., J. Cell Sci. 96:207-217, 1990).

Experimental data indicated that the novel alpha6p variant contained a significant alteration in the heavy chain, which is entirely extracellular. The current structural model of the subunit proposes that the seven N-terminal repeats adopt the fold of a beta-propeller domain (Oxvig and Springer, Proc. Natl. Acad. Sci. U.S.A. 95:4870-4875, 1998; Springer, Proc. Natl. Acad. Sci. U.S.A. 94:65-72, 1997). These domains contain seven four-stranded beta-sheets and are arranged in a torus around a pseudosymmetric axis. Structural homology studies of enzymes with known beta-propeller folds have identified active sites at the top of the beta-propeller, typically where adjacent loops run in opposite directions (Fulop, et al., Cell 94:161-170, 1998; Callebaut and Mornon, Cell Mol. Life Sci. 54:880-891, 1998; Paoli et al., Nat. Struct. Biol. 6:926-931, 1999). Recent studies of the beta-propeller domain in integrins have demonstrated that folds 1 and 3 in the alpha4 integrin subunit are important for ligand binding (Irie et al., Proc. Natl. Acad. Sci. U.S.A. 94:7198-7203, 1997), whereas the alpha5 integrin ligand binding site is determined by amino acid sequences in repeats 2 and 3 of the N-terminal domain of the subunit (Mould et al., J. Biol. Chem. 275:20324-20336, 2000). The mass spectrometry data described herein, which indicated that the alpha6p variant contained only exons 13-25, indicates that the entire proposed beta-propeller domain is missing. Thus, the alpha6p integrin variant would function as an inactive receptor for cellular adhesion to the extracellular ligand. The production of this alpha subunit variant on the cell surface may be a mechanism for regulation of extracellular adhesion.

Additionally, because integrins are known to be conformationally dependent molecules with dynamic ligand interactions (Humphries, Curr. Opin. Cell Biol. 8:632-640, 1996), alteration of the extracellular portion of the molecule could influence intracellular signaling (Filardo and Cheresh, J. Biol. Chem. 269:4641-4647, 1994). The integrin alpha subunit cytoplasmic domains have been shown to be important for a diverse number of functions including adhesion, motility, internalization, differentiation, and cytoskeletal organization (Price et al., J. Exp. Med. 186:1725-1735,1997; Shaw and Mercurio, J. Cell Biol. 123:1017-1025, 1993; Kawaguchi and Hemler, J. Biol. Chem. 268:16279-16285, 1993; Lu and Springer, J. Immunol. 159:268-278, 1997; Gaietta et al., J. Cell Sci. 107:3339-3349, 1994; Honda et al., J. Clin. Endocrinol. Metab. 80:2899-2905, 1995). Recently, the role of the alpha6A cytoplasmic domain was examined in myoblasts and found to inhibit proliferation and promote differentiation. Interestingly, the cytoplasmic tail alone suppressed signaling through the focal adhesion kinase and mitogen-activated protein kinase pathways (Sastry et al., J. Cell Biol. 144:1295-1309, 1999).

Experimental data indicated that the altered extracellular region of the alpha6p variant did not affect its ability to remain paired with either alpha4 or beta1 integrin subunits. The alpha6p variant was immunoprecipitated using the anti-alpha4 integrin monoclonal antibody, A9, whose epitope is present when alpha6 is coupled to the beta4 subunit (Van Waes et al., Cancer Res. 51:2395-2402, 1991). This finding suggests that the alpha6p subunit is able to heterodimerize with the alpha4 subunit in the same manner as the full-length alpha6 integrin. It is also noteworthy that alpha6p co-immunoprecipitated with beta1 integrin in DU145H cells but not in HaCaT cells, despite abundant levels of beta1 integrin in the HaCaT cells (FIG. 3B).

Previous studies suggested that integrins and TM4 tetraspan proteins could interact with one another to modulate integrin signaling and adhesion (Ikeyama et al., J. Exp. Med. 177:1231-1237, 1993; Berditchevski and Odintsova, J. Cell Biol. 146:477-492, 1999). It has also been demonstrated that two members of this family, CD9 and CD81, can interact with the extracellular domain of the alpha6 integrin (Berditchevski et al., Mol. Biol. Cell 7:193-207, 1996).

Information obtained from cell surface retention half-life studies revealed that alpha6p (70 kDa) was almost three times more stable than that of the full-length alpha6 form (FIG. 8). This data indicated that the alpha6p protein was not a degradation product of the full-length alpha6 integrin because the protein was not preferentially cleared from the surface as would be expected for a protein targeted for degradation. The alpha6p protein was not generated after cell lysis, because multiple antiproteases and short immunoprecipitation times were unable to alter the presence of this variant. Although some integrins are highly susceptible to proteolytic processing, e.g., the beta4 integrin (von Bredow et al., Exp. Cell Res. 236:341-345, 1997), the fully processed alpha6 integrin has not yet been reported to be enzymatically cleaved by any enzymes in vivo. Proteolytic cleavage of the alpha6 integrin in vivo could not be produced in previous studies (von Bredow et al., Exp. Cell Res. 236:341-345, 1997).

The experimental data does not suggest that alpha6p originated from an alternative splicing event, because analysis by RT-PCR revealed that only one transcript for alpha6 was present within the known coding region (FIG. 9). Moreover, it has not previously been demonstrated in humans that alternative splicing plays a role in the regulation of the extracellular domain of integrins (Sonnenberg, Curr. Top. Microbiol. Immunol. 184:7-35, 1993). Several integrins including alpha6 have been shown to have isoforms of the cytoplasmic domain generated by alternative splicing (Tamura, J. Cell Biol. 111:1593-1604, 1990; Tamura, Proc. Natl. Acad. Sci. U.S.A. 88:10183-10187, 1991; Cooper et al., J. Cell Biol. 115:843-850, 1991). The experimental data demonstrated a significant variation (a 70-kDa change) in the extracellular heavy chain of the alpha6p integrin (FIG. 2). This large extracellular variation has not been described previously for other integrins.

Taken together, the experimental data suggest that a post-transcriptional event is responsible for the generation of alpha6p. The alpha6 integrin subunit, in addition to other subunits, normally undergoes endoproteolytic processing close to the C terminus after synthesis, resulting in the formation of a light and heavy chain (Berthet et al., J. Biol. Chem. 275:33308-33313, 2000). A previous report demonstrated that defective post-transcriptional processing of the pre-alpha6 transcript in carcinoma cells lead to loss of normal cleavage and a resulting larger 150-kDa single protein (Delwel et al., Biochem. J. 324:263-272, 1997). Examples of normal post-transcriptional processing have been described in yeast via translational introns that can give rise to two different sized proteins from a single mRNA transcript (Engelberg-Kulka et al., Trends Biochem. Sci. 18:294-296, 1993). Alternatively, ribosomal scanning past the conventional initiation codon has been described for major histocompatability class I molecules. In this process, the ribosome initiates translation further downstream (Bullock and Eisenlohr, J. Exp. Med. 184:1319-1329, 1996). Twelve alternative initiation codons are predicted within the alpha6 gene and one (position 1833) precedes exon 13.

Other embodiments of the invention are within the following claims.

I claim:

1. An isolated polypeptide comprising a truncated alpha6 integrin extracellular domain, wherein the N-terminal amino acid of the truncated alpha6 integrin corresponds to an amino acid between amino acid residues 517 and 597 of the full-length alpha6 integrin.

2. The isolated polypeptide of claim 1, wherein the amino acid sequence of the truncated alpha6 integrin is at least 98% identical to SEQ ID NO:1 in the region C-terminal to amino acid 597.

3. The isolated polypeptide of claim 2, wherein the N-terminal amino acid of the truncated alpha6 integrin corresponds to one of amino acids 571-596 of SEQ ID NO:1.

4. The isolated polypeptide of claim 2, wherein the N-terminal amino acid of the polypeptide corresponds to one of amino acids 588-596 of SEQ ID NO:1.

5. An isolated polypeptide comprising a truncated alpha6 integrin, wherein the amino acid sequence corresponding to amino acids 1-516 of SEQ ID NO:1 is absent.

6. An isolated polypeptide comprising a fragment of an alpha6 integrin, wherein the N-terminal amino acid of the fragment corresponds to an amino acid encoded by a sequence that lies within exon 12 or 13 of the full-length alpha6 integrin, and the isolated polypeptide does not include a sequence corresponding to amino acids 1-516 of SEQ ID NO:1.

7. The isolated polypeptide of claim 1, wherein the polypeptide is protease-resistant.

8. An isolated polypeptide, the polypeptide comprising a naturally occurring fragment of an alpha6 integrin, wherein the N-terminal amino acid of the naturally occurring alpha6 fragment begins with an amino acid encoded by a sequence within exons 12-13 of SEQ ID NO:2.

9. The polypeptide of claim 1, wherein the isolated polypeptide is alpha6p.

10. The polypeptide of claim 1, wherein the N-terminus of the polypeptide sequence begins with the amino acid sequence RVN.

11. The polypeptide of claim 1, wherein the polypeptide is isolated from a human cell.

12. The polypeptide of claim 1, wherein the polypeptide is not recognized by an antibody that binds an epitope located between amino acids 1 and 500 of SEQ ID NO:1.

13. The polypeptide of claim 1, wherein the polypeptide can bind at least one antibody in the group consisting of GoH3, J1B5, 4F10, AA6A, and BQ16; and wherein the polypeptide does not substantially bind an A33 polyclonal antibody.

14. The polypeptide of claim 1, further comprising a heterologous, non-integrin amino acid sequence.

15. The polypeptide of claim 1 further comprising a transmembrane domain.

16. The polypeptide of claim 8, wherein the N-terminus of the polypeptide corresponds to the N-terminus of the alpha6 variant present on DU145H cells.

17. A method of producing a truncated alpha6 integrin, the method comprising culturing a host cell that contains a heterologous nucleic acid that encodes the polypeptide of claim 1 under conditions in which the heterologous nucleic acid is expressed.

18. The method of claim 17, wherein the heterologous nucleic acid encodes a protease-resistant fragment of alpha6 integrin.

19. An isolated nucleic acid comprising a sequence encoding a polypeptide, wherein the polypeptide comprises a naturally occurring fragment of an alpha6 integrin, and wherein the N-terminal amino acid of the polypeptide corresponds to an amino acid between amino acid residues 517 and 597 of the full-length alpha6 integrin.

20. The isolated nucleic acid of claim 19, wherein the N-terminal amino acid of the polypeptide corresponds to one of amino acids 571-596 of SEQ ID NO:1.

21. The isolated nucleic acid of claim 19, wherein the N-terminal amino acid of the polypeptide corresponds to one of amino acids 588-596 of SEQ ID NO:1.

22. The isolated nucleic acid of claim 19, wherein the polypeptide is alpha6p.

23. The isolated nucleic acid of claim 19, further comprising a vector nucleic acid sequence.

24. The isolated nucleic acid of claim 23, wherein the vector nucleic acid sequence comprises a promoter and terminator sequence operably linked to the sequence encoding the polypeptide.

25. The isolated nucleic acid of claim 23, wherein the vector nucleic acid sequence comprises a selectable marker gene.

27. The isolated nucleic acid of claim 20, wherein the nucleic acid hybridizes to the nucleic acid sequence of nucleotides 1711 to 1788 of SEQ ID NO:2.

28. The isolated nucleic acid of claim 20, wherein the nucleic acid comprises a sequence that differs by fewer than five substitutions, insertions, or deletions from the nucleic acid sequence of nucleotides 1711 to 1788 of SEQ ID NO:2.

29. A host cell that contains a heterologous nucleic acid comprising the nucleic acid of claim 19.

30. The host cell of claim 2, which is a mammalian host cell.


SEQUENCE LISTING (7 sequences)

SEQ ID NO: 1 - protein, 1073 aa, Homo sapiens
MAAAGQLCLLYLSAGLLSRLGAAFNLDTREDNVIRKYGDPGSLFGFSLAMHWQLQPEDKRLLLVGAPRGEALPLQRANRTGGLYSCDITARGPCTRIEFDNDADPTSESKEDQWMGVTVQSQGPGGKVVTCAHRYEKRQHVNTKQESRDIFGRCYVLSQNLRIEDDMDGGDWSFCDGRLRGHEKFGSCQQGVAATFTKDFHYIVFGAPGTYNWKGIVRVEQKNNTFFDMNIFEDGPYEVGGETEHDESLVPVPANSYLGFSLDSGKGIVSKDEITFVSGAPRANHSGAVVLLKRDMKSAHLLPEHIFDGEGLASSFGYDVAVVDLNKDGWQDIVIGAPQYFDRDGEVGGAVYVYMNQQGRWNNVKPIRLNGTKDSMFGIAVKNIGDINQDGYPDIAVGAPYDDLGKVFIYHGSANGINTKPTQVLKGISPYFGYSIAGNMDLDRNSYPDVAVGSLSDSVTIFRSRPVINIQKTITVTPNRIDLRQKTACGAPSGICLQVKSCFEYTANPAGYNPSISIVGTLEAEKERRKSGLSSRVQFRNQGSEPKYTQELTLKRQKQKVCMEETLWLQDNIRDKLRPIPITASVEIQEPSSRRRVNSLPEVLPILNSDEPKTAHIDVHFLKEGCGDDNVCNSNLKLEYKFCTREGNQDKFSYLPIQKGVPELVLKDQKDIALEITVTNSPSNPRNPTKDGDDAHEAKLIATFPDTLTYSAYRELRAFPEKQLSCVANQNGSQADCELGNPFKRNSNVTFYLVLSTTEVTFDTPYLDINLKLETTSNQDNLAPITAKAKVVIELLLSVSGVAKPSQVYFGGTVVGEQAMKSEDEVGSLIEYEFRVINLGKPLTNLGTATLNIQWPKEISNGKWLLYLVKVESKGLEKVTCEPQKEINSLNLTESHNSRKKREITEKQIDDNRKFSLFAERKYQTLNCSVNVNCVNIRCPLRGLDSKASLILRSRLWNSTFLEEYSKLNYLDILMRAFIDVTAAAENIRLPNAGTQVRVTVFPSKTVAQYSGVPWWIILVAILAGILMLALLVFILWKCGFFKRNKKDHYDATYHKAEIHAQPSDKERLTSDA

SEQ ID NO: 2 - DNA, 3222 bp, Homo sapiens
  CDS (1)...(3219)
atggccgccgccgggcagctgtgcttgctctacctgtcggcggggctcctgtcccggctcggcgcagccttcaacttggacactcgggaggacaacgtgatccggaaatatggagaccccgggagcctcttcggcttctcgctggccatgcactggcaactgcagcccgaggacaagcggctgttgctcgtgggggccccgcgggcagaagcgcttccactgcagagagccaacagaacgggagggctgtacagctgcgacatcaccgcccgggggccatgcacgcggatcgagtttgataacgatgctgaccccacgtcagaaagcaaggaagatcagtggatgggggtcaccgtccagagccaaggtccagggggcaaggtcgtgacatgtgctcaccgatatgaaaaaaggcagcatgttaatacgaagcaggaatcccgagacatctttgggcggtgttatgtcctgagtcagaatctcaggattgaagacgatatggatgggggagattggagcttttgtgatgggcgattgagaggccatgagaaatttggctcttgccagcaaggtgtagcagctacttttactaaagactttcattacattgtatttggagccccgggtacttataactggaaagggattgttcgtgtagagcaaaagaataacactttttttgacatgaacatctttgaagatgggccttatgaagttggtggagagactgagcatgatgaaagtctcgttcctgttcctgctaacagttacttaggtttttctttggactcagggaaaggtattgtttctaaagatgagatcacttttgtatctggtgctcccagagccaatcacagtggagccgtggttttgctgaagagagacatgaagtctgcacatctcctccctgagcacatattcgatggagaaggtctggcctcttcatttggctatgatgtggcggtggtggacctcaacaaggatgggtggcaagatatagttattggagccccacagtattttgatagagatggagaagttggaggtgcagtgtatgtctacatgaaccagcaaggcagatggaataatgtgaagccaattcgtcttaatggaaccaaagattctatgtttggcattgcagtaaaaaatattggagatattaatcaagatggctacccagatattgcagttggagctccgtatgatgacttgggaaaggtttttatctatcatggatctgcaaatggaataaataccaaaccaacacaggttctcaagggtatatcaccttattttggatattcaattgctggaaacatggaccttgatcgaaattcctaccctgatgttgctgttggttccctctcagattcagtaactattttcagatcccggcctgtgattaatattcagaaaaccatcacagtaactcctaacagaattgacctccgccagaaaacagcgtgtggggcgcctagtgggatatgcctccaggttaaatcctgttttgaatatactgctaaccccgctggttataatccttcaatatcaattgtgggcacacttgaagctgaaaaagaaagaagaaaatctgggctatcctcaagagttcagtttcgaaaccaaggttctgagcccaaatatactcaagaactaactctgaagaggcagaaacagaaagtgtgcatggaggaaaccctgtggctacaggataatatcagagataaactgcgtcccattcccataactgcctcagtggagatccaagagccaagctctcgtaggcgagtgaattcacttccagaagttcttccaattctgaattcagatgaacccaagacagctcatattgatgttcacttcttaaaagagggatgtggagacgacaatgtatgtaacagcaaccttaaactagaatataaattttgcacccgagaaggaaatcaagacaaattttcttatttaccaattcaaaaaggtgtaccagaactagttctaaaagatcagaaggatattgctttagaaataacagtgacaaacagcccttccaacccaaggaatcccacaaaagatggcgatgacgcccatgaggctaaactgattgcaacgtttccagacactttaacctattctgcatatagagaactgagggctttccctgagaaacagttgagttgtgttgccaaccagaatggctcgcaagctgactgtgagctcggaaatccttttaaaagaaattcaaatgtcactttttatttggttttaagtacaactgaagtcacctttgacaccccatatctggatattaatctgaagttagaaacaacaagcaatcaagataatttggctccaattacagctaaagcaaaagtggttattgaactgcttttatcggtctcgggagttgctaaaccttcccaggtgtattttggaggtacagttgttggcgagcaagctatgaaatctgaagatgaagtgggaagtttaatagagtatgaattcagggtaataaacttaggtaaacctcttacaaacctcggcacagcaaccttgaacattcagtggccaaaagaaattagcaatgggaaatggttgctttatttggtgaaagtagaatccaaaggattggaaaaggtaacttgtgagccacaaaaggagataaactccctgaacctaacggagtctcacaactcaagaaagaaacgggaaattactgaaaaacagatagatgataacagaaaattttctttatttgctgaaagaaaataccagactcttaactgtagcgtgaacgtgaactgtgtgaacatcagatgcccgctgcgggggctggacagcaaggcgtctcttattttgcgctcgaggttatggaacagcacatttctagaggaatattccaaactgaactacttggacattctcatgcgagccttcattgatgtgactgctgctgccgaaaatatcaggctgccaaatgcaggcactcaggttcgagtgactgtgtttccctcaaagactgtagctcagtattcgggagtaccttggtggatcatcctagtggctattctcgctgggatcttgatgcttgctttattagtgtttatactatggaagtgtggtttcttcaagagaaataagaaagatcattatgatgccacatatcacaaggctgagatccatgctcagccatctgataaagagaggcttacttctgatgcatag

SEQ ID NO: 3 - protein, 16 aa, Homo sapiens
CIHAQPSDKERLTSDA

SEQ ID NO: 4 - protein, 4 aa, Homo sapiens
RRVN

SEQ ID NO: 5 - protein, 5 aa, Homo sapiens
RRRVN

SEQ ID NO: 6 - protein, 6 aa, Homo sapiens
SRRRVN

SEQ ID NO: 7 - protein, 7 aa, Homo sapiens
SSRRRVN